(19) United States
(12) Patent Application Publication
(10) Pub. No.: US 2011/0110977 A1
(43) Pub. Date: May 12, 2011

(54) Multiclade HIV Vaccines

(76) Inventors: Susan W. Barnett, San Francisco, CA (US); Raul Victor Gomez-Roman, Copenhagen (DK); Brian Burke, Lafayette, CA (US); Ying Lian, Vallejo, CA (US); Indresh K. Srivastava, Concord, MA (US)

(21) Appl. No.: 12/083,905

(22) PCT Filed: Oct. 17, 2006
(86) PCT No.: PCT/US2006/041023

§ 371 Date: Nov. 8, 2010

Related U.S. Application Data
(60) Provisional application No. 60/727,665, filed on Oct. 17, 2005.
(60) Provisional application No. 60/754,466, filed on Dec. 27, 2005.

(51) Int. Cl. A61K 39/21; A61P 31/18

(52) U.S. Cl. 424/208.1

(57) ABSTRACT

Compositions comprising multivalent and adjuvanted HIV Env glycoproteins are described. Methods of using these compositions for treatment and prevention of HIV are also provided.

### RELATED APPLICATIONS

This application claims the benefit of U.S. Provisional Application No. 60/727,665, filed Oct. 17, 2005, and U.S. Provisional Application No. 60/754,466, filed Dec. 27, 2005. The teachings of the above applications are incorporated herein in their entirety by reference.

### STATEMENT OF FEDERAL FUNDING

The invention was supported, in whole or in part, by NIAID-NIH HIVRAD Grant No. 5P01 AI48225-03 from the National Institute of Allergy and Infectious Diseases. The Government may have certain rights in the invention.

### TECHNICAL FIELD

The invention relates generally to compositions comprising multivalent HIV envelope (Env) glycoproteins. The invention also pertains to methods of using these compositions to elicit an immune response, for example to elicit a broadly neutralizing antibody response.

### BACKGROUND OF THE INVENTION

One of the most ravaging diseases of the late twentieth century has been acquired immunodeficiency syndrome (AIDS), caused by infection with HIV (see, e.g., Barre-Sinoussi et al. (1983) Science 220:868-871; Gallo et al. (1984) Science 224:500-503; Levy et al. (1984) Science 225:840-842; Siegal et al. (1981) N Engl. J. Med. 305:1439-1444). There are several known strains of HIV including HIV-1, a collective term referring to several strains isolated in Europe or America, and HIV-2, a strain endemic in many West African countries. HIV-1 (also referred to as HTLV-III, LAV or HTLV-III/LAV) is classified by phylogenetic analysis into three groups, group M (major), group O (outlier) and a variant of HIV-1, designated group N, that has been identified with its epicenter in Cameroon (Simon et al. (1998) Nat. Med. 4:1032-1037). All three HIV-1 groups cause AIDS.

AIDS patients usually have a long asymptomatic period followed by the progressive degeneration of the immune system and the central nervous system. Replication of the virus is highly regulated, and both latent and lytic infection of the CD4 positive helper subset of T-lymphocytes occur in tissue culture (Zagury et al. (1986) Science 231:850-853). Molecular studies of HIV-1 show that it encodes a number of genes (Ratner et al. (1985) Nature 313:277-284; Sanchez-Pescador et al. (1985) Science 227:484-492), including three structural genes—gag, pol and env—that are common to all retroviruses. Nucleotide sequences from viral genomes of other retroviruses, particularly HIV-2 and simian immunodeficiency viruses, SIV (previously referred to as STLV-III), also contain these structural genes (Guyader et al. (1987) Nature 326:662-669).

The envelope protein of HIV-1, HIV-2 and SIV is a glycoprotein of about 160 kd (gp160). During virus infection of the host cell, gp160 is cleaved by host cell proteases to form gp120 and the integral membrane protein, gp41. The gp41 portion is anchored in the membrane bilayer of virion, while the gp120 segment protrudes into the surrounding environment. gp120 and gp41 are more covalently associated and free gp120 can be released from the surface of virions and infected cells.

Crystallography studies of gp120 liganded to CD4 and/or co-receptors indicate that this polypeptide is folded into two major domains having certain emanating structures. The inner domain (inner with respect to the N and C terminus) features a two-helix, two-stranded bundle with a small five-stranded.-sandwich at its termini-proximal end and a projection at the distal end from which the V1/V2 stem emanates. The outer domain is a staked double barrel that lies along side the inner domain so that the outer barrel and inner bundle axes are approximately parallel. Between the distal inner domain and the distal outer domain is a four-stranded bridging sheet that holds a peculiar minidomain in contact with, but distinct from, the inner, the outer domain, and the V1/V2 domain. The bridging sheet is composed of four β-strand structures (β-3, β-2, -β21, β-20). The bridging region is packed primarily over the inner domain, although some surface residues of the outer domain, such as Phe 382, reach into the bridging sheet to form part of its hydrophobic core. See, also WO 00/39303. As recently demonstrated by Chen et al. (2005) Nature 433:834-841, Env glycoproteins (e.g., SIV Env) exhibit very different structures when unliganded.

Immunogenicity of HIV Env polypeptides has also been studied but broadly neutralizing antibody responses have not been observed. See, e.g., Javaherian, K. et al. (1989) Proc. Natl. Acad. Sci. USA 86:6786-6772; Matsushita, M. et al. (1988) J. Virol. 62:2107-2144; Putney, S. et al. (1986) Science 234:1392-1395; Rushe, J. R. et al. (1988) Proc. Nat. Acad. Sci. USA 85: 3198-3202; Matthews, T. (1986) Proc. Natl. Acad. Sci. USA 83:9709-9713; Nara, P. L. et al. (1988) J. Virol. 62:2622-2628; Palker, T. J. et al. (1988) Proc. Natl. Acad. Sci. USA 85:1932-1936); Stamatatos et al. (1998) AIDS Res Hum Retroviruses 14(13):1129-1139; Wyatt, R. et al. (1995) J. Virol. 69:5723-5733; Leu et al. (1998) AIDS Res. and Human Retroviruses 14:151-155.

Therefore, there remains a need for compositions that can elicit broad neutralizing antibody responses in a subject.

### SUMMARY OF THE INVENTION

The present invention relates to multivalent HIV envelope (Env) compositions. In a particular embodiment, the HIV Env composition comprises two or more (at least two) HIV envelope polypeptides, wherein at least two of the envelope polypeptides are each from different HIV subtypes. In another embodiment, the HIV Env composition comprises three or more (at least three) HIV envelope polypeptides, wherein at least three of the envelope polypeptides are each from different HIV subtypes. In a further embodiment, the HIV Env compositions comprise one are more adjuvants (MF59, CpG molecules, microparticles such as PLG microparticles, alum, etc.). In a particular embodiment, the HIV Env compositions comprise an immunopotentiator molecule such as CpG. Thus, in certain embodiments, the invention relates to multivalent compositions comprising two or more (at least two) adjuvanted HIV Env glycoproteins.

In other embodiments, the multivalent HIV Env composition comprises two or more (at least two) HIV envelope polypeptides, wherein at least two of the envelope polypeptides are each from different HIV types (e.g., HIV-1, HIV-2). In still other embodiments, the multivalent HIV Env composition comprises two or more (at least two) HIV envelope polypeptides, wherein at least two of the envelope polypeptides are each from different strains from the same subtypes (e.g., HIV-1_{SF2}, HIV-1_{SF162}, HIV-1_{CM235}, etc).

In certain embodiments, the HIV Env glycoprotein comprises a gp120. In other embodiments, the HIV Env glycoprotein comprises gp140. In yet other embodiments, the HIV Env glycoprotein comprises a gp160. In any of the compositions described herein, the HIV Env glycoprotein can be expressed in a monomeric or oligomeric form. In a particular embodiment, the HW Env glycoprotein comprises an oligomeric gp140 (o-gp140). In another embodiment, the HIV Env glycoprotein comprises an oligomeric gp140 comprising a deletion of a portion of the V2 loop. In other embodiments, the HIV Env glycoprotein comprises an oligomeric gp140 polypeptide comprising a deletion of a portion of the V1 loop, an oligomeric gp140 polypeptide comprising, a deletion of a portion of the V3 loop or an oligomeric gp 140 polypeptide with a mutated protease cleavage site.

In particular embodiments, the multivalent compositions described herein include HIV Env polypeptides from two or more subtypes selected from the group consisting of subtypes A (e.g., A1, A2), B, C, D, F (e.g., F1, F2), G, H, J and K: In other embodiments, the multivalent compositions described herein also include HIV Env polypeptides from two or more subtypes selected from the group consisting of subtypes A (e.g., A1, A2), B, C, D, F (e.g., F1, F2), G, H, J and K and circulating recombinant forms (CRFs). In still other embodiments, the multivalent compositions described herein further include HIV Env polypeptides from two or more subtypes and/or CRFs. CRFs have also been referred to in the art, as well as herein, as subtypes E and I. Thus, the multivalent compositions can comprise HIV Env glycoproteins from two or more of the subtypes or CRFs, for example, HIV Env glycoproteins from 2, 3, 4, 5, 6, 7, 8, 9 or 10 subtypes or CRFs. In certain embodiments, the multivalent compositions comprise HIV Env glycoproteins from subtypes A and B. In other embodiments, the multivalent compositions comprise HIV Env glycoproteins from subtypes A and C. In still further embodiments, the multivalent compositions comprise HIV Env glycoproteins from subtypes B and C. In other embodiments, the multivalent compositions comprise HIV Env glycoproteins from subtypes A, B and C. In yet other embodiments, the multivalent compositions comprise HIV Env glycoproteins from subtypes A, B and E. In other embodiments, the multivalent compositions comprise HIV Env glycoproteins from subtypes A and B; A and C; A and E; B and C; B and E; or C and E.

In certain embodiments, the HIV Env glycoproteins are complexed to one or more additional molecules (ligands) selected from the group consisting of CD4, CD4 mimetics, CCR5 co-receptor or mimetic, tat, other viral proteins, polynucleotide, polypeptide, small molecules and combinations thereof.

In a particular embodiment, the multivalent HIV Env glycoprotein compositions described herein include one or more adjuvants (e.g., MF59, CpG molecules, microparticles such as PLG microparticles, alum, etc.). In certain embodiments, the adjuvant comprises MF59. In other embodiments, the adjuvant comprises one or more CpG molecules. In still other embodiments, the adjuvant comprises MF59 and one or more CpG molecules. In yet other embodiments, the adjuvant comprises alum and/or one or more microparticles (e.g., PLG microparticles).

In another aspect, polynucleotides encoding any of the polypeptides described herein are provided. In certain embodiments, the polynucleotides are carried on gene delivery vehicles, for example aplasmid, a viral vector (e.g., adenovirus vector, poxvirus vector, alphavirus vector, etc.) or non-viral delivery vector.

In another aspect, immunogenic compositions and vaccine compositions comprising any of the polypeptides, polynucleotides and/or gene delivery vehicles described herein are provided.

In yet another aspect, a method of inducing an immune response (e.g., an innate, a humoral response such as a neutralizing antibody response and/or a cellular immune response) in subject comprising, administering any of the compositions, polynucleotides and/or polypeptides described herein to a subject in an amount sufficient to induce an immune response in the subject. In certain embodiments, the methods comprise administering a first composition comprising any of the polynucleotides described herein in a priming step and (b) administering a second composition comprising any of the adjuvanted Env glycoproteins described herein, as a booster, in an amount sufficient to induce an immune response in the subject. In any of these methods, the polynucleotides may be delivered as DNA (e.g., plasmids) or using viral (e.g., adenovirus, poxvirus and/or alphavirus) or non-viral vectors. In certain preferred embodiments, the priming step comprises administering one or more alphavirus and/or poxvirus and/or adenovirus vectors comprising polynucleotides as described herein and the boosting step comprises administering one or more multivalent HIV Env glycoprotein-containing compositions described herein. Furthermore, in any of these methods the composition may elicit an immune response that is protective against HIV infection from various strains. In certain embodiments, the methods described herein induce a protective immune response to the subtypes represented by the HIV Env glycoproteins in the composition. Preferably, the methods described herein induce a protective immune response to subtypes represented by the multivalent Env glycoproteins and, in addition, induce a protective immune response to strains from at least one subtype (e.g., 1, 2, 3, 4, 5, 6, 7, 8, 9, 10 or even more subtypes) not represented in the multivalent composition.

In yet another aspect, the invention includes a method of increasing the potency, durability and/or breadth of an immune response in a subject by administering any of the compositions, polynucleotides and/or polypeptides described herein to a subject in an amount sufficient to induce an immune response in the subject. In certain embodiments, the methods comprise administering a first composition comprising any of the polynucleotides described herein in a priming step and (b) administering a second composition comprising any of the adjuvanted Env glycoproteins described herein, as a booster, in an amount sufficient to induce an immune response in the subject. In any of these methods, the polynucleotides may be delivered as formulated and unformulated DNA (e.g., plasmids with or without carriers) or using viral (e.g., adenovirus and/or poxvirus and/or alphavirus) or non-viral vectors. In certain preferred embodiments, the priming step comprises administering one or more alphavirus and/or adenovirus vectors comprising polynucleotides as described herein and the boosting step comprises administering one or more multivalent HIV Env glycoprotein-containing compositions described herein.

Thus, in certain embodiments, methods of inducing a protective immune response are described, for example, in Which the compositions protect against strains from each of the subtypes from which the HIV Env glycoproteins of the multivalent composition are derived. In addition, the compositions may also protect against strains from subtypes not represented in the administered composition. For example, methods of inducing a protective immune response in a subject to HIV strains from subtypes A, B and C may be achieved using an adjuvanted multivalent HIV Env glycoprotein containing HIV Env glycoproteins derived from a subtype B strain and a subtype C strain; methods of inducing a protective immune response in a subject to HIV strains from subtypes A, B, C and E may be achieved using an adjuvanted multivalent HIV Env glycoprotein containing HIV Env glycoproteins derived from a subtype B strain and a subtype C strain or a subtype B strain and a subtype E strain; etc.

These and other embodiments of the subject invention will readily occur to those of skill in the art in light of the disclosure herein.

### BRIEF DESCRIPTION OF THE DRAWINGS

FIGS. 1A and 1B shows a table depicting neutralization of HIV subtype B by sera obtained from rabbits immunized with adjuvanted HIV Env glycoprotein compositions at two weeks post third and two weeks post fourth immunizations. Shading or boxed shading indicates >50% or >80% virus neutralization in the strains tested, respectively.

FIG. 2 is a graph depicting neutralizing antibody titers against HIV SF162 strain after immunization of rabbits with the various adjuvanted gp140-containing compositions indicated below the bars. For each composition, titers are shown prebleed (light, left bar), two weeks after a third immunization (gray, middle bar) and two weeks after a fourth immunization (dark, right bar).

FIG. 3 is a graph depicting 80% geomean neutralizing titers (GMT) of HIV SF162 strain by sera obtained from rabbits after immunization with the various adjuvanted gp140-containing compositions indicated below the bars. For each composition, antibody titers are shown prebleed, two weeks after a second immunization, two weeks after a third immunization, two weeks after a fourth immunization and six months post fourth immunization. The results show that high neutralizing antibody titers to SF162 were elicited in all groups.

FIG. 4 are graphs depicting 80% geomean neutralizing titers (GMT) of HIV SF162 strain by sera obtained from rabbits after immunization with the various adjuvanted Env glycoprotein-containing compositions indicated. Results are shown two weeks post second immunization, two weeks post third immunization and two weeks post fourth immunization. The results show that adjuvanting with both MF59 and CpG enhanced neutralizing antibody responses against SF162.

FIG. 5 is a graph depicting 80% neutralizing antibody titers of HIV SF162 strain by sera obtained from rabbits after immunization with the various adjuvanted Env glycoprotein-containing compositions indicated along the horizontal axis. Results are shown two weeks post fourth immunization. The results show that significant enhancement of neutralizing antibody responses were elicited in CpG immunized groups.

FIG. 6 is a graph depicting 80% neutralizing antibody titers of HIV SF162 strain by sera obtained from rabbits after immunization with the various adjuvanted Env glycoprotein-containing compositions indicated along the horizontal axis. Results are shown two weeks post fourth immunization. The results show that bivalent immunization in MF59 enhanced neutralizing antibody titers to SF162.

FIG. 7 is a graph depicting 80% neutralizing antibody titers of HIV SF162 strain by sera obtained from rabbits after immunization with the various adjuvanted Env glycoprotein-containing compositions indicated along the horizontal axis. Results are shown two weeks post fourth immunization. The results show that bivalent immunization in MF59 plus CpG significantly enhanced neutralizing antibody titers to SF162 over TV1 alone.

FIG. 8 is a table depicting P values for the comparison of geomean neutralization titers against SF162 for the immunization groups indicated at the left over time. P values were calculated as in FIG. 5. Shading indicates a significant difference between the groups. Results are shown two weeks post second immunization, two weeks post third immunization, two weeks post fourth immunization and six months post fourth immunization. The results show significant differences in group geomean titers (GMT) to HIV SF162 strain were observed as early as two weeks post second immunization.

FIG. 9 is a graph depicting 50% neutralizing antibody titers of HIV SF162 strain by sera obtained from rabbits after immunization with the various adjuvanted Env glycoprotein-containing compositions indicated along the horizontal axis. Results are shown prebleed, two weeks post second immunization, two weeks post third immunization and two weeks post fourth immunization. Background titer levels are 25 for 2wp3 sera and 20 for the remaining sera samples. The results show that TV 1.21 neutralizing antibody responses are elicited only in CpG-containing groups.

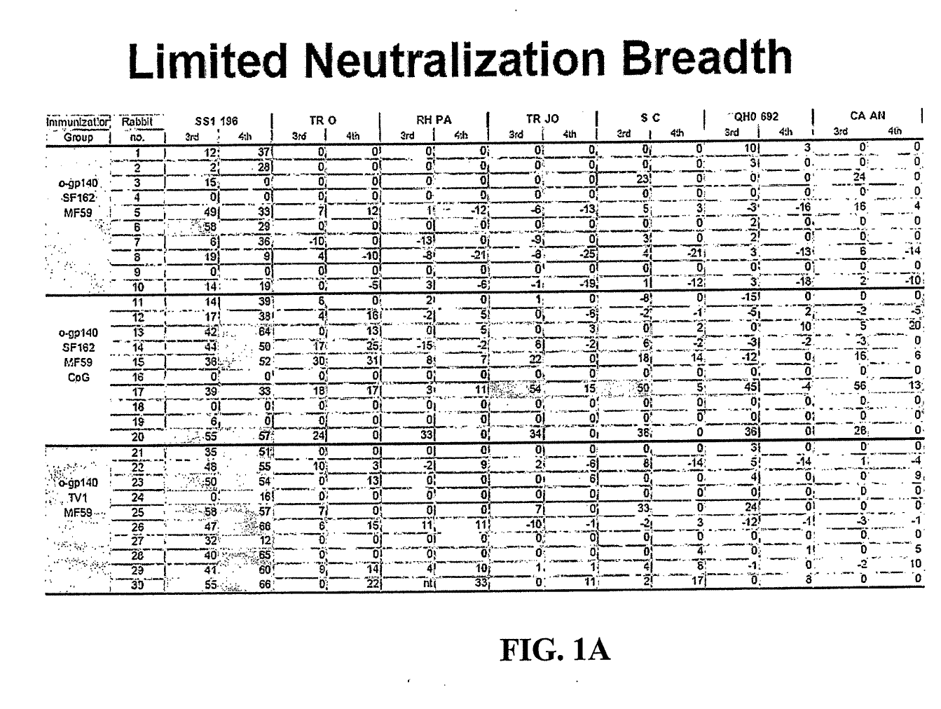

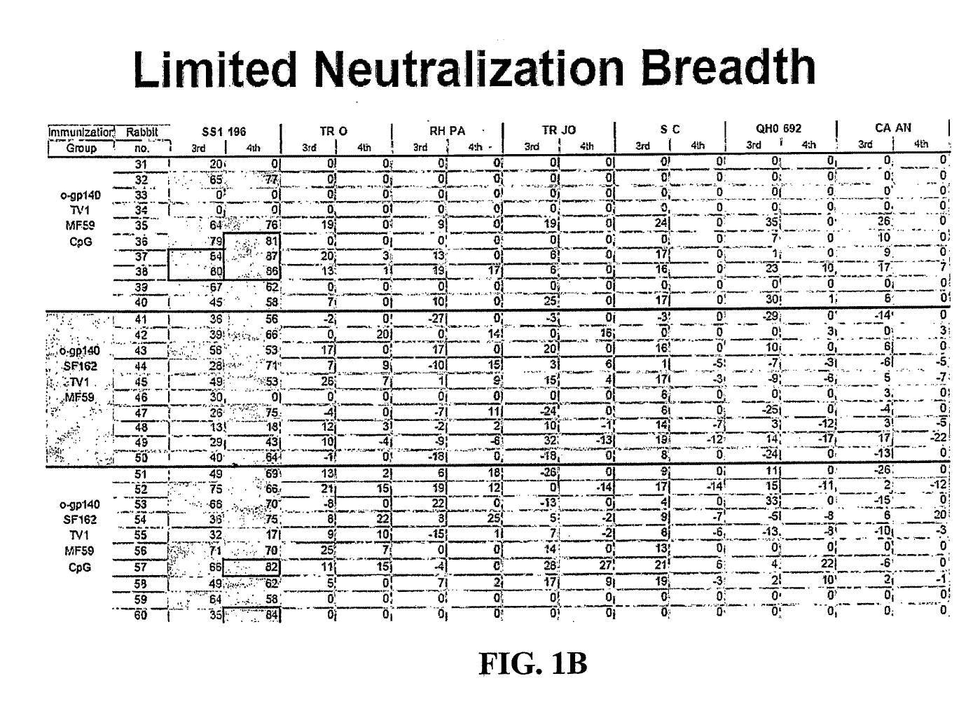

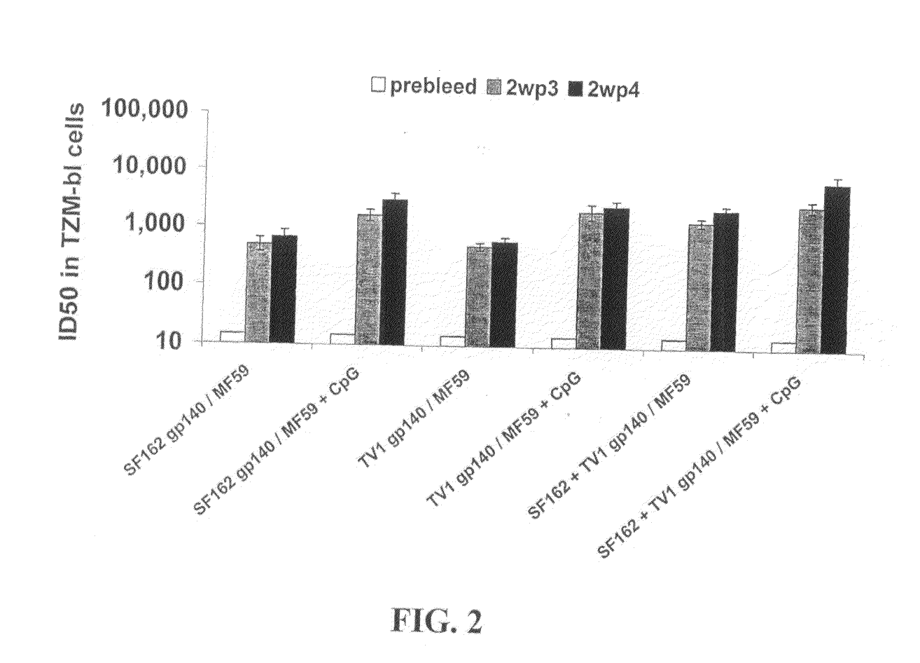

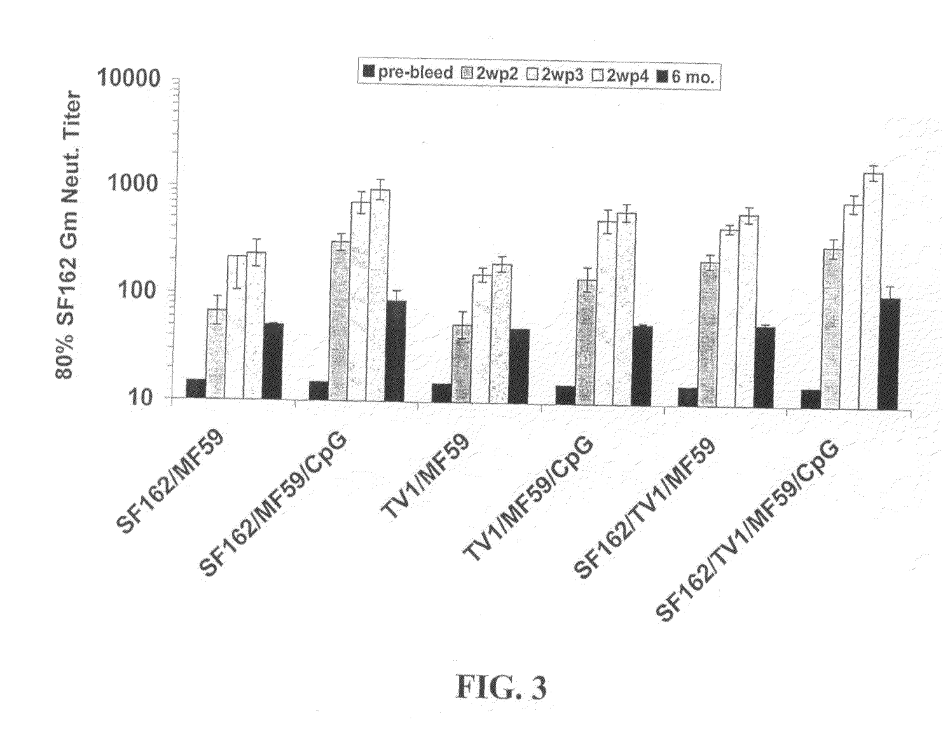

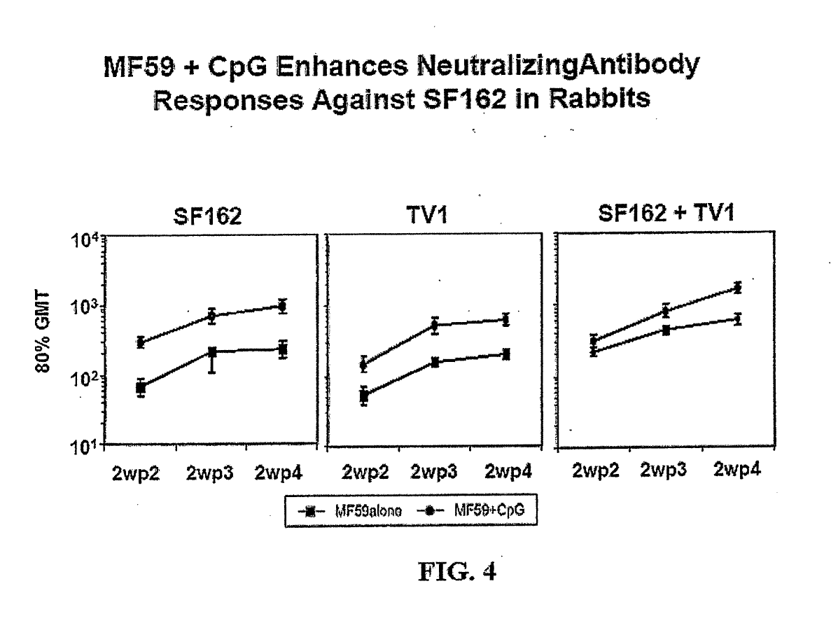

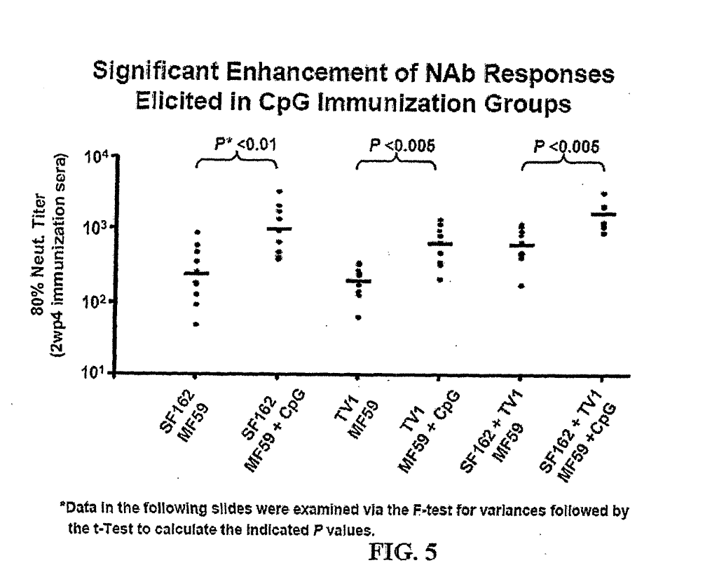

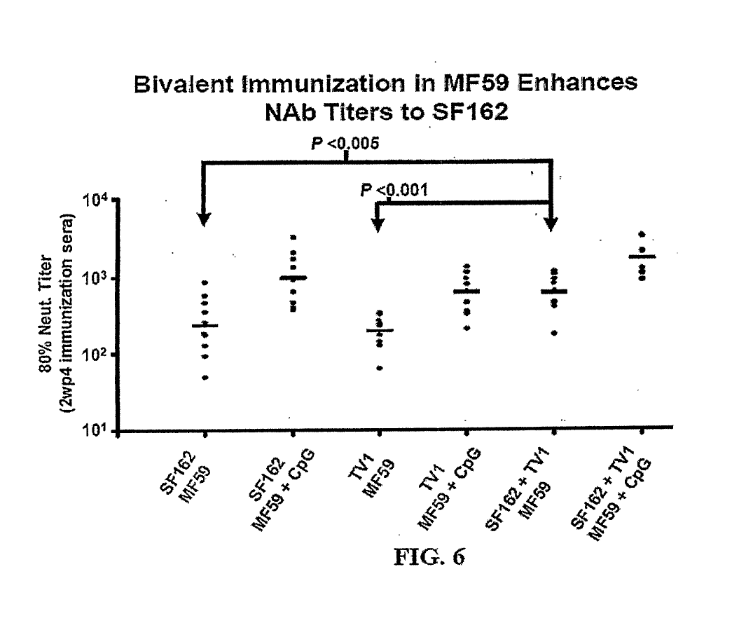

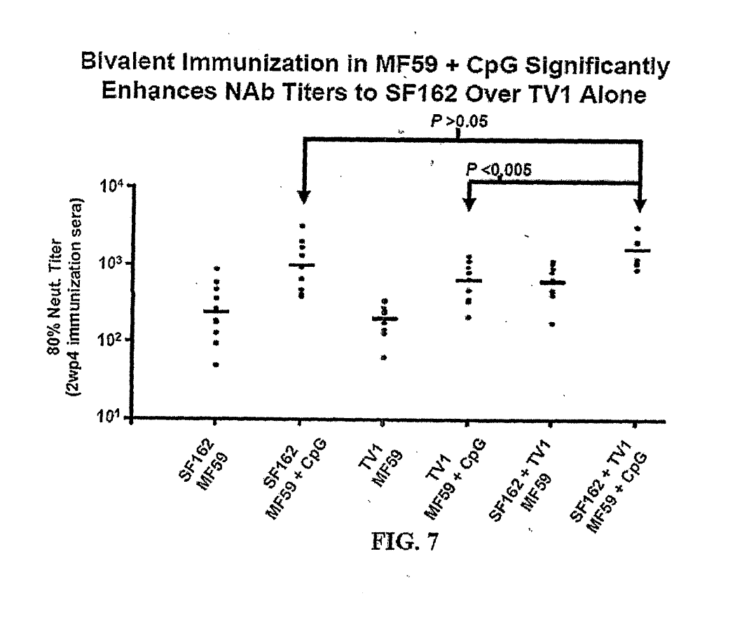

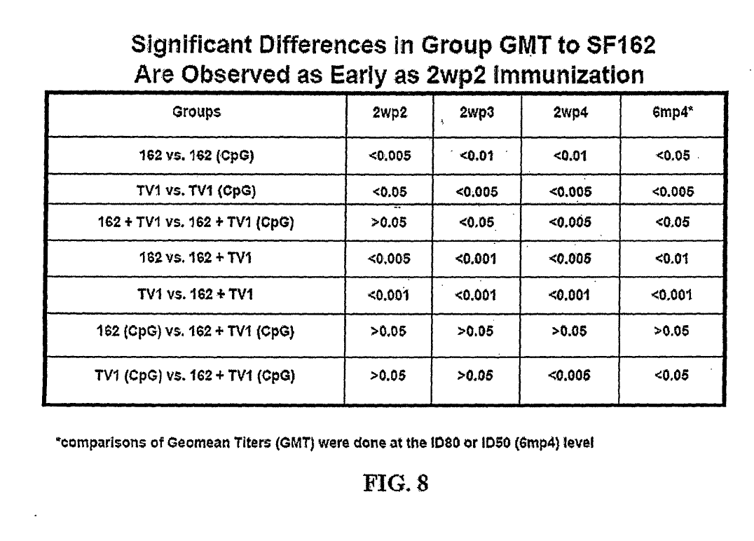

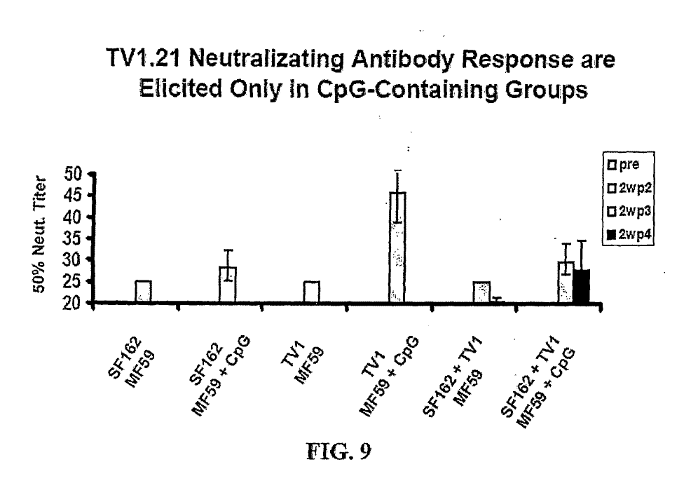

### DETAILED DESCRIPTION OF THE INVENTION

The practice of the present invention will employ, unless otherwise indicated, conventional methods of protein chemistry, viral immunobiology, molecular biology and recombinant DNA techniques within the skill of the art. Such techniques are explained fully in the literature. See, e.g., T. E. Creighton, Proteins: Structures and Molecular Properties (W. H. Freeman and Company, 1993); Nelson L. M. and Jerome H. K., HIV Protocols in Methods in Molecular Medicine, vol. 17, 1999; Sambrook, J. et al., Molecular Cloning: A Laboratory Manual, 3rd edition (Cold Spring Harbor Laboratory Press, 2001); Ausubel, F. M. et al. (eds.), Short Protocols in Molecular Biology, 5th ed. (Current Protocols, 2002); and Lipkowitz and Boyd, Reviews in Computational Chemistry, volumes 1-present (Wiley-VCH, New York, N.Y., 1999).

It must be noted that, as used in this specification and the appended claims, the singular forms “a”, “an” and “the” include plural referents unless the content clearly dictates otherwise. Thus, for example, reference to “a polypeptide” includes a mixture of two or more polypeptides, and the like.

All publications, patents and patent applications cited herein, whether supra or infra, are hereby incorporated by reference in their entirety.

Definitions

In describing the present invention, the following terms will be employed, and are defined as indicated below.

The terms “polypeptide,” and “protein” are used interchangeably herein to denote any polymer of amino acid residues. The terms encompass peptides, oligopeptides, dimers, multimers, and the like. Such polypeptides can be derived from natural sources or can be synthesized or recombinantly produced. The terms also include postexpression modifications of the polypeptide, for example, glycosylation, acetylation, phosphorylation, etc.

A polypeptide as defined herein is generally made up of the 20 natural amino acids Ala (A), Arg (R), Mn (N), Asp (D), Cys (C), Gln (Q), Glu (E), Gly (G), His (H), Ile (I), Leu (L), Lys (K), Met (M), Phe (F), Pro (P), Ser (S), Thr (T), Trp (W), Tyr (Y) and Val (V) and may also include any of the several known amino acid analogs, both naturally occurring and synthesized analogs, such as but not limited to homoisoleucine, asaleucine, 2-(methylenecyclo-propyl)glycine, S-methylcysteine, S-(prop-1-enyl)cysteine, homoserine, ornithine, norleucine, norvaline, homoarginine, 3-(3-carboxyphenyl)alanine, cyclohexylalanine, mimosine, pipecolic acid, 4-methylglutamic acid, canavanine, 2,3-diaminopropionic acid, and the like. Further examples of polypeptide agents which will find use in the present invention are set forth below.

By “geometry” or “tertiary structure” of a polypeptide or protein is meant the overall 3-D configuration of the protein. As described herein, the geometry can be determined, for example, by crystallography studies or by using various programs or algorithms which predict the geometry based on interactions between the amino acids making up the primary and secondary structures.

By “wild type” polypeptide, polypeptide agent or polypeptide drug, is meant a naturally occurring polypeptide sequence, and its corresponding secondary structure. An “isolated” or “purified” protein or polypeptide is a protein which is separate and discrete from a whole organism with which the protein is normally associated in nature. It is apparent that the term denotes proteins of various levels of purity. Typically, a composition containing a purified protein will be one in which at least about 35%, preferably at least about 40-50% (40%, 45%, 50%), more preferably, at least about 75-85% (e.g., 75%, 80%, 85%), and most preferably at least about 90%, 91%, 92%, 93%, 94%, 95%, 96%, 97%, 98%, 99%, 99.5% or more, of the total protein in the composition will be the protein in question.

HIV-1 is classified by phylogenetic analysis into three groups: group M (major), group O (outlier) and a variant of HIV-1, designated group N. Subtypes (clades) represent different lineages of HIV and have geographic associations. Subtypes of HIV-1 are phylogenetically associated groups of HIV-1 sequences, with the sequences within any one subtype or sub-subtype more similar to each other than to sequences from different subtypes throughout their genomes. See, e.g., Los Alamos National Laboratory HIV Sequence Database (http://hiv-web.lanl.gov/content/hiv-db/HelpDocs/subtypes-more.html) (Los Alamos, N.Mex.).

The HIV-1 M group subtypes are phylogenetically associated groups or clades of HIV-1 sequences, and include subtypes A (e.g., A1, A2), B, C, D, F (e.g., F1, F2), G, H, J and K. Subtypes and sub-subtypes of the HIV-1 M group are thought to have diverged in humans, following a single chimpanzee-to-human transmission event. The worldwide distribution of various HIV-1 M group subtypes is diverse, with subtype B being most prevalent in North America and Europe and subtype A being most prevalent in Africa. Whereas most subtypes are common in Central Africa, other areas have restricted distribution of genotypes. For example, subtype C is common in India and South Africa, and subtype F is prevalent in Romania, Brazil and Argentina. The HIV-1 M group also includes circulating recombinant forms (CRFs), which are viruses whose complete genome is a recombinant or mosaic consisting of some regions which cluster with one subtype and other regions of the genome which cluster with another subtype in phylogenetic analyses. Examples of CRFs are found in the Los Alamos National Laboratory HIV Sequence Database (http://www.hiv.1an1.gov/content/hiv-db/mainpage.html) (Los Alamos, N.Mex.). CRFs have also been referred to in the art, as well as herein, as subtypes E and I. CRFs (subtype E) are highly prevalent in Thailand.

The HIV-1 O group includes most divergent viruses that do not cluster with group M strains. Type O infections have been identified in the West Central African countries of Cameroon and neighboring countries, such as Equatorial Guinea and Gabon. The spread of group O infections to Europe and more recently to the United States has been documented, although all patients have had links to West Central Africa. The HIV-1 O group is thought to be the result of a separate chimpanzee-to-human transmission event, with intra-group diversification into the “subtype” clades resulting in the human population after each transfer event.

The HIV-1 N group (also referred to as the “new” group) includes viruses that are distinct from HIV-1 groups M and O. The HIV-1 N group is also thought to be the result of a separate chimpanzee-to-human transmission event, with intra-group diversification into the “subtype” clades resulting in the human population after each transfer event.

HIV-2 is classified into five clades: A, B, C, F and G clades.

By “Env polypeptide” is meant a molecule derived from an envelope protein, preferably from HIV Env. The term includes Env polypeptides and polynucleotides encoding Env polypeptides. The envelope protein of HIV-1 is a glycoprotein of about 160 kd (gp160). During virus infection of the host cell, gp160 is cleaved by host cell proteases to form gp120 and the integral membrane protein, gp41. The gp41 portion is anchored in (and spans) the membrane bilayer of virion, while the gp120 segment protrudes into the surrounding environment. As there is no covalent attachment between gp120 and gp41, free gp120 is released from the surface of virions and infected cells. Env polypeptides may also include gp140 polypeptides. Env polypeptides (gp120, gp140, etc.) can exist as monomers, dimers or multimers (oligomers such as trimers).

By “oligomeric gp140 polypeptide” or “o-gp140” is meant any oligomeric form of gp140 polypeptide. Oligomeric forms of gp140 include an o-gp140 comprising a deletion of a portion of the V1 loop, an o-gp140 polypeptide comprising a deletion of a portion of the V2 loop, an o-gp140 polypeptide comprising a deletion of a portion of the V3 loop, an o-gp140 polypeptide with a mutated protease cleavage site. Oligomeric-gp140 glycoproteins may adopt a configuration that mimics the native, trimeric env spikes present on the surface of the HIV virion and, accordingly, may be desirable for immunogenic compositions. See, e.g., Yang et al. (2000) J. Virol. 74(12):5716-5725; Grundner et al. (2005) Virology 331(1):33-46; Srivastava et al. (2003) J. Virol. 77(29):11244-11259; Barnett et al. (2001) J. Virol. 75(12):5526-5540; WO 00/39302; U.S. Pat. No. 6,602,705; Srivastava et al. (2002) J. Virol. 76(6):2835-2847.

Furthermore, an “Env polypeptide” as defined herein is not limited to a polypeptide having the exact sequences described herein. Indeed, the HIV genome is in a state of constant flux and contains several variable domains which exhibit relatively high degrees of variability between isolates. It is readily apparent that the terms encompass Env (e.g., o-gp140) polypeptides from any of the identified HIV isolates, as well as newly identified isolates, and subtypes of these isolates. Descriptions of structural features are given herein with reference to HXB-2. One of ordinary skill in the art in view of the teachings of the present disclosure and the art can determine corresponding regions in other HIV variants (e.g., isolates HIV_{IIIb}, HIV_{SF2}, HIV-1_{SF162}, HIV-1_{SF170}, HIV_{LAV}, HIV_{LAI}, HIV_{MN}, HIV-1_{CM235}, HIV-1_{US4}, other HIV-1 strains from diverse subtypes, HIV-2 strains and diverse subtypes (e.g., HIV-2_{UC1} and HIV-2_{UC2}), and simian immunodeficiency virus (SIV). (See, e.g., Virology, 3rd Edition (W. K. Joklik ed. 1988); Fundamental Virology, 2nd Edition (B. N. Fields and D. M. Knipe, eds. 1991); Fields, B. N. et al. (eds.), Fields Virology, 4th edition (Lippincott Williams & Wilkins, 2001), for a description of these and other related viruses), using for example, sequence comparison programs (e.g., BLAST and others described herein) or identification and alignment of structural features (e.g., a program such as the “ALB” program described herein that can identify β-sheet regions). The actual amino acid sequences of the modified Env polypeptides can be based on any HIV variant.

Additionally, the term “Env polypeptide” encompasses proteins that include additional modifications as compared to the native sequence, such as additional internal deletions, additions and substitutions. These modifications may be deliberate, as through site-directed mutagenesis, or may be accidental, such as through naturally occurring mutational events. Modifications of Env include, but are not limited to, generating polynucleotides that encode Env polypeptides having mutations and/or deletions therein. For instance, some or all of hypervariable regions, V1, V2, V3, V4 and/or V5 can be deleted or modified, particularly regions V1, V2, and V3. V1 and V2 regions may mask CCR5 co-receptor binding sites (see, e.g., Moulard et al. (2002) Proc. Natl. Acad. Sci. USA 14:9405-9416). Accordingly, in certain embodiments, some or all of the variable loop regions are deleted, for example to expose potentially conserved neutralizing epitopes. Further, deglycosylation of N-linked sites are also potential targets for modification inasmuch as a high degree of glycosylation also serves to shield potential neutralizing epitopes on the surface of the protein. Additional optional modifications, used alone or in combination with variable region deletes and/ordeglycosylation modification, include modifications (e.g., deletions) to the beta-sheet regions (e.g., as described in WO 00/39303), modifications of the leader sequence (e.g., addition of Kozak sequences and/or replacing the modified wild type leader with a native or sequence-modified tpa leader sequence) and/or modifications to protease cleavage sites (see, e.g., Chakrabarti et al. (2002) J. Virol. 76(11):5357-5368 describing a gp140 Delta CFI containing deletions in the cleavage site, fusogenic domain of gp41, and spacing of heptad repeats 1 and 2 of gp41 that retained native antigenic conformational determinants as defined by binding to known monoclonal antibodies or CD4, oligomer formation, and virus neutralization in vitro). If the Env polypeptide is to be used in vaccine compositions, the modifications must be such that immunological activity (i.e., the ability to elicit an antibody response to the polypeptide) is not lost. Similarly, if the polypeptides are to be used for diagnostic purposes, such capability must be retained.

Examples of such Env polypeptides include, but are not limited to, the following: a gp140 comprising a deletion of a portion of the V1 loop, a gp140 polypeptide comprising a deletion of a portion of the V2 loop, a gp140 polypeptide comprising a deletion of a portion of the V3 loop, a gp140 polypeptide with a mutated protease cleavage site, a gp160 comprising a deletion of a portion of the V1 loop, a gp160 polypeptide comprising a deletion of a portion of the V2 loop, a gp160 polypeptide comprising a deletion of a portion of the V3 loop, and a gp160 polypeptide with a mutated protease cleavage site.

Env polypeptides may be recombinantly produced in host cells. These polypeptides may be secreted into growth medium in which an organism expressing the protein is cultured. Alternatively, such polypeptides may also be recovered intracellularly. Secretion into growth media is readily determined using a number of detection techniques, including, e.g., polyacrylamide gel electrophoresis and the like, and immunological techniques such as Western blotting and immunoprecipitation assays as described in, e.g., International Publication No. WO 96/04301. Env polypeptides such as trimeric o-gp140 polypeptides, for example, be produced and/or purified as described in Srivastava et al. (2002) J. Virol. 76(6):2835-2847 and Srivastava et al. (2003) J. Virol. 77(29):11244-11259.

An “immunogenic” Env glycoprotein is a molecule (Env polypeptide or polynucleotide encoding an Env polypeptide) that includes (or encodes) at least one epitope such that the molecule is capable of either eliciting an immunological reaction in an individual to which the protein is administered or, in the diagnostic context, is capable of reacting with antibodies directed against the HIV in question.

By “multivalent” or “polyvalent” is meant a composition or vaccine that includes at least two (i.e., two or more) of the same or different HIV peptide(s). In a particular embodiment, the HIV peptides are from different HIV types (e.g., HIV-1, HIV-2, etc), different HIV subtypes (e.g., HIV-1 subtype A, HIV-1 subtype B, HIV-1 subtype C, etc) or different strains from the same subtype (e.g., HIV-1_{SF2}, HIV-1_{SF162}, etc). For example, a multivalent composition or vaccine, as described herein, includes compositions comprising HIV Env glycoproteins from two or more different HIV types, strains and/or subtypes. The term “subtypes” includes the subtypes currently identified as well as circulating recombinant forms (CRFs). HIV subtypes (including CRFs) are continually being characterized and can be found on the HIV database from Los Alamos National Laboratories, available on the internet. Thus, a multivalent vaccine can include peptides derived from two or more different subtypes (including A (e.g., A1, A2), B, C, D, E, F (e.g., F1, F2), G, H, J and K, as well as various CRFs), for example HIV Env glycoproteins derived from subtypes A and B; A and C; B and C; A and E; B and E; C and E; A, B and C; A, B and E, etc. Similarly, the term “bivalent” refers to a composition or vaccine that includes two of the same or different envelope peptide(s). In a particular embodiment, a bivalent composition or vaccine, as described herein, includes two HIV Env glycoproteins from different HIV types, different HIV subtypes or different strains from the same subtype. In a more particular embodiment, a bivalent composition or vaccine, as described herein, comprises two HIV Env glycoproteins from different subtypes (e.g., subtypes B and C, subtypes B and E, etc).

By “epitope” is meant a site on an antigen to which specific B cells and/or T cells respond, rendering the molecule including such an epitope capable of eliciting an immunological reaction or capable of reacting with HIV antibodies present in a biological sample. The term is also used interchangeably with “antigenic determinant” or “antigenic determinant site.” An epitope can comprise three (3) or more amino acids in a spatial conformation unique to the epitope. Generally, an epitope consists of at least five (5) such amino acids and, more usually, consists of at least 8-10 such amino acids. Methods of determining spatial conformation of amino acids are known in the art and include, for example, x-ray crystallography and two-dimensional nuclear magnetic resonance. Furthermore, the identification of epitopes in a given protein is readily accomplished using techniques well known in the art, such as by the use of hydrophobicity studies and by site-directed serology. See, also, Geysen et al. (1984) Proc. Natl. Acad. Sci. USA 81:3998-4002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al. (1986) Molecular Immunology 23:709-715 (technique for identifying peptides with high affinity for a given antibody). Antibodies that recognize the same epitope can be identified in a simple immunoassay showing the ability of one antibody to block the binding of another antibody to a target antigen.

An “immunological response” or “immune response” to an antigen or composition is the development in a subject of an innate, humoral and/or a cellular immune response to an antigen present in the composition of interest.

The term “antibody” as used herein includes antibodies obtained from both polyclonal and monoclonal preparations, as well as, the following: (i) hybrid (chimeric) antibody molecules (see, for example, Winter et al. (1991) Nature 349:293-299; and U.S. Pat. No. 4,816,567); (ii) F(ab′)₂ and F(ab) fragments; (iii) Fv molecules (noncovalent heterodimers, see, for example, Inbar et al. (1972) Proc. Natl. Acad. Sci. USA 69:2659-2662; and Ehrlich et al. (1980) Biochem. 19:4091-4096); (iv) single-chain Fv molecules (sFv) (see, for example, Huston et al. (1988) Proc. Natl. Acad. Sci. USA 85:5879-5883); (v) dimeric and trimeric antibody fragment constructs; (vi) humanized antibody molecules (see, for example, Riechmann et al. (1988) Nature 332:323-327; Verhoeyan et al. (1988) Science 239:1534-1536; and U.K. Patent Publication No. GB 2,276,169, published Sep. 21, 1994); (vii) Mini-antibodies or minibodies (i.e., sFv polypeptide chains that include oligomerization domains at their C-termini, separated from the sFv by a hinge region; see, e.g., Pack et al. (1992) Biochem. 31:1579-1584; Cumber et al. (1992) J. Immunol. 149B:120-126); and, (vii) any functional fragments obtained from such molecules, wherein such fragments retain specific-binding properties of the parent antibody molecule.

Thus, the term “antibody” refers to a polypeptide or group of polypeptides which comprise at least one antigen binding site. An “antigen binding site” is formed from the folding of the variable domains of an antibody molecule(s) to form three-dimensional binding sites with an internal surface shape and charge distribution complementary to the features of an epitope of an antigen, which allows specific binding to form an antibody-antigen complex. An antigen binding site may be formed from a heavy- and/or light-chain domain (V_{H} and V_{L}, respectively), which form hypervariable loops which contribute to antigen binding. The term “antibody” includes, without limitation, polyclonal antibodies, monoclonal antibodies, chimeric antibodies, altered antibodies, univalent antibodies, Fab proteins, and single-domain antibodies. In many cases, the binding phenomena of antibodies to antigens is equivalent to other ligand/anti-ligand binding.

If polyclonal antibodies are desired, a selected mammal (e.g., mouse, rabbit, goat, horse, non-human primates, humans, etc.) is immunized with an immunogenic polypeptide bearing an HIV epitope(s). Serum from the immunized animal is collected and treated according to known procedures. If serum containing polyclonal antibodies to an HIV Env glycoprotein epitope contains antibodies to other antigens, the polyclonal antibodies can be purified by immunoaffinity chromatography. Techniques for producing and processing polyclonal antisera are known in the art, see for example, Mayer and Walker, eds. (1987) Immunochemical Methods In Cell and Molecular Biology (Academic Press, London).

Monoclonal antibodies directed against HIV Env glycoprotein epitopes can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. See, e.g., M. Schreier et al. (1980) Hybridoma Techniques; Hammerling et al. (1981) Monoclonal Antibodies and T-Cell Hybridomas; Kennett et al. (1980) Monoclonal Antibodies; U.S. Pat. Nos. 4,341,761; 4,399,121; 4,427,783; 4,444,887; 4,466,917; 4,472,500; 4,491,632; and 4,493,890. Panels of monoclonal antibodies produced against HIV epitopes can be screened for various properties; i.e., for isotype, epitope affinity, etc. As used herein, a “single domain antibody” (dAb) is an antibody which is comprised of an H_{L} domain, which binds specifically with a designated antigen. A dAb does not contain a V_{L} domain, but may contain other antigen binding domains known to exist to antibodies, for example, the kappa and lambda domains. Methods for preparing dabs are known in the art. See, for example, Ward et al. (1989) Nature 341:544-546.

Antibodies can also be comprised of V_{H} and V_{L} domains, as well as other known antigen binding domains. Examples of these types of antibodies and methods for their preparation are known in the art (see, e.g., U.S. Pat. No. 4,816,467). For example, “vertebrate antibodies” refer to antibodies which are tetramers or aggregates thereof, comprising light and heavy chains which are usually aggregated in a “Y” configuration and which may or may not have covalent linkages between the chains. In vertebrate antibodies, the amino acid sequences of the chains are homologous with those sequences found in antibodies produced in vertebrates, whether in situ or in vitro (for example, in hybridomas). Vertebrate antibodies include, for example, purified polyclonal antibodies and monoclonal antibodies, methods for the preparation of which are described infra.

“Hybrid antibodies” are antibodies where chains are separately homologous with reference to mammalian antibody chains and represent novel assemblies of them, so that two different antigens are precipitable by the tetramer or aggregate. In hybrid antibodies, one pair of heavy and light chains are homologous to those found in an antibody raised against a first antigen, while a second pair of chains are homologous to those found in an antibody raised against a second antibody. This results in the property of “divalence”, i.e., the ability to bind two antigens simultaneously. Such hybrids can also be formed using chimeric chains, as set forth below.

“Chimeric antibodies” refer to antibodies in which the heavy and/or light chains are fusion proteins. Typically, one portion of the amino acid sequences of the chain is homologous to corresponding sequences in an antibody derived from a particular species or a particular class, while the remaining segment of the chain is homologous to the sequences derived from another species and/or class. Usually, the variable region of both light and heavy chains mimics the variable regions or antibodies derived from one species of vertebrates, while the constant portions are homologous to the sequences in the antibodies derived from another species of vertebrates. However, the definition is not limited to this particular example. Also included is any antibody in which either or both of the heavy or light chains are composed of combinations of sequences mimicking the sequences in antibodies of different sources, whether these sources be from differing classes or different species of origin, and whether or not the fusion point is at the variable/constant boundary. Thus, it is possible to produce antibodies in which neither the constant nor the variable region mimic known antibody sequences. It then becomes possible, for example, to construct antibodies whose variable region has a higher specific affinity for a particular antigen, or whose constant region can elicit enhanced complement fixation, or to make other improvements in properties possessed by a particular constant region.

Another example is “altered antibodies”, which refer to antibodies in which the naturally occurring amino acid sequence in a vertebrate antibody has been varies. Utilizing recombinant DNA techniques, antibodies can be redesigned to obtain desired characteristics. The possible variations are many, and range from the changing of one or more amino acids to the complete redesign of a region, for example, the constant region. Changes in the constant region, in general, to attain desired cellular process characteristics, e.g., changes in complement fixation, interaction with membranes, and other effector functions. Changes in the variable region can be made to alter antigen binding characteristics. The antibody can also be engineered to aid the specific delivery of a molecule or substance to a specific cell or tissue site. The desired alterations can be made by known techniques in molecular biology, e.g., recombinant techniques, site-directed mutagenesis, etc.

Yet another example are “univalent antibodies”, which are aggregates comprised of a heavy-chain/light-chain dimer bound to the Fc (i.e., stem) region of a second heavy chain. This type of antibody escapes antigenic modulation. See, e.g., Glennie et al. (1982) Nature 295:712-714. Included also within the definition of antibodies are “Fab” fragments of antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab′), as well as tetramers containing the 2H and 2L chains (referred to as F(ab)₂), which are capable of selectively reacting with a designated antigen or antigen family. Fab antibodies can be divided into subsets analogous to those described above, i.e., “vertebrate Fab”, “hybrid Fab”, “chimeric Fab”, and “altered Fab”. Methods of producing Fab fragments of antibodies are known within the art and include, for example, proteolysis, and synthesis by recombinant techniques.

“Antigen-antibody complex” refers to the complex formed by an antibody that is specifically bound to an epitope on an antigen.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively.

Two or more polynucleotide sequences can be compared by determining their “percent identity.” Two or more amino acid sequences likewise can be compared by determining their “percent identity.” The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov (1986) Nucl. Acids Res. 14(6):6745-6763. An implementation of this algorithm for nucleic acid and peptide sequences is provided by the Genetics Computer Group (Madison, Wis.) in their BestFit utility application. The default parameters for this method are described in the Wisconsin Sequence Analysis Package Program Manual, Version 8 (1995) (available from Genetics Computer Group, Madison, Wis.). Other equally suitable programs for calculating the percent identity or similarity between sequences are generally known in the art.

For example, percent identity of a particular nucleotide sequence to a reference sequence can be determined using the homology algorithm of Smith and Waterman with a default scoring table and a gap penalty of six nucleotide positions. Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith-Waterman algorithm can be employed where default parameters are used for the scoring table (for example, gap open penalty of 12, gap extension penalty of one, and a gap of six). From the data generated, the “Match” value reflects “sequence identity.” Other suitable programs for calculating the percent identity or similarity between sequences are generally known in the art, such as the alignment program BLAST, which can also be used with default parameters. For example, BLASTN and BLASTP can be used with the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

One of skill in the art can readily determine the proper search parameters to use for a given sequence in the above programs. For example, the search parameters may vary based on the size of the sequence in question. Thus, for example, a representative embodiment of the present invention would include an isolated polynucleotide having X contiguous nucleotides, wherein (i) the X contiguous nucleotides have at least about 50% identity to Y contiguous nucleotides derived from any of the sequences described herein, (ii) X equals Y, and (iii) X is greater than or equal to 6 nucleotides and up to 5000 nucleotides, preferably greater than or equal to 8 nucleotides and up to 5000 nucleotides, more preferably 10-12 nucleotides and up to 5000 nucleotides, and even more preferably 15-20 nucleotides, up to the number of nucleotides present in the full-length sequences described herein (e.g., see the Sequence Listing and claims), including all integer values falling within the above-described ranges.

The synthetic expression cassettes (and purified polynucleotides) of the present invention include related polynucleotide sequences having about 80% to 100%, greater than 80-85% (e.g., greater than 80%, 81%, 82%, 83%, 84% or 85%), preferably greater than 90-92% (e.g., greater than 90%, 91% or 92%), more preferably greater than 95% (e.g., greater than 95%, 96% or 97%), and most preferably greater than 98% (e.g., greater than 98%, 99%, 99.5% or more) sequence (including all integer values falling within these described ranges) identity to the synthetic expression cassette sequences disclosed herein (for example, to the claimed sequences or other sequences of the present invention) when the sequences of the present invention are used as the query sequence.

Computer programs are also available to determine the likelihood of certain polypeptides to form structures such as β-sheets. One such program, described herein, is the “ALB” program for protein and polypeptide secondary structure calculation and predication. In addition, secondary protein structure can be predicted from the primary amino acid sequence, for example using protein crystal structure and aligning the protein sequence related to the crystal structure (e.g., using Molecular Operating Environment (MOE) programs available from the Chemical Computing Group Inc., Montreal, P.Q., Canada). Other methods of predicting secondary structures are described, for example, in Gamier et al. (1996) Methods Enzymol. 266:540-553; Geourjon et al. (1995) Comput. Applic. Biosci. 11:681-684; Levin (1997) Protein Eng. 10:771-776; and Rost et al. (1993) J. Molec. Biol. 232:584-599.

Homology can also be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85% (e.g., at least about 80%, 81%, 82%, 83%, 84% or 85%), preferably at least about 90%, and most preferably at least about 95%-98% (e.g., at least about 95%, 96%, 97% or 98%) sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

A “coding sequence” or a sequence that “encodes” a selected protein, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to cDNA from viral nucleotide sequences as well as synthetic and semisynthetic DNA sequences and sequences including base analogs. A transcription termination sequence may be located 3′ to the coding sequence.

“Control elements” refers collectively to promoter sequences, ribosome binding sites, polyadenylation signals, transcription termination sequences, upstream regulatory domains, enhancers, and the like, which collectively provide for the transcription and translation of a coding sequence in a host cell. Not all of these control elements need always be present so long as the desired gene is capable of being transcribed and translated.

A control element “directs the transcription” of a coding sequence in a cell when RNA polymerase will bind the promoter sequence and transcribe the coding sequence into mRNA, which is then translated into the polypeptide encoded by the coding sequence.

“Operably linked” refers to an arrangement of elements wherein the components so described are configured so as to perform their usual function. Thus, control elements operably linked to a coding sequence are capable of effecting the expression of the coding sequence when RNA polymerase is present. The control elements need not be contiguous with the coding sequence, so long as they function to direct the expression thereof. Thus, for example, intervening untranslated yet transcribed sequences can be present between, e.g., a promoter sequence and the coding sequence and the promoter sequence can still be considered “operably linked” to the coding sequence.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with whichit is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting procaryotic microorganisms or eucaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

By “vertebrate subject” is meant any member of the subphylum chordata, including, without limitation, humans and other primates, including non-human primates such as chimpanzees, rhesus macaques, baboons and other apes and monkey species; farm animals such as cattle, sheep, pigs, goats and horses; domestic mammals such as dogs and cats; laboratory animals including rodents such as mice, rats, rabbits and guinea pigs; birds, including domestic, wild and game birds such as chickens, turkeys and other gallinaceous birds, ducks, geese, and the like. The term does not denote a particular age. Thus, both adult and newborn individuals are intended to be covered.

As used herein, a “biological sample” refers to a sample of tissue or fluid isolated from an individual, including but not limited to, for example, blood, plasma, serum, fecal matter, urine, bone marrow, bile, spinal fluid, lymph fluid, samples of the skin, external secretions of the skin, respiratory, intestinal, and genitourinary tracts, samples derived from the gastric epithelium and gastric mucosa, tears, saliva, milk, blood cells, organs, biopsies and also samples of in vitro cell culture constituents including but not limited to conditioned media resulting from the growth of cells and tissues in culture medium, e.g., recombinant cells, and cell components.

Overview

The present invention relates to compositions comprising at least two HIV envelope glycoproteins (and, optionally, one or more adjuvants) and the use of these compositions, for example to elicit neutralizing antibody responses against more than one HIV, strain, type or subtype. The compositions described herein are multivalent in that they include HIV Env polypeptides from more than one HIV strain, type, subtype and/or isolate. Such multivalent compositions elicit an immune response in a subject and, in certain embodiments, may be used to broaden and/or enhance the immune response elicited in the subject as compared to a univalent composition. Different isolates represented in the multivalent compositions may represent different viral serotypes, and may utilize different modes of entry into the host cell (e.g., coreceptors). By “broaden” or “enhance” is meant an immune response that is greater in magnitude (e.g., additive or synergistic) and/or results in greater neutralizing (antiviral) activity against a more diverse array of HIV isolates than that of any of the single component Env polypeptides that comprise the multivalent composition. Various forms of the different embodiments of the invention, described herein, may be combined.

Env Polypeptides

The Env polypeptide portion of the complexes described herein can be derived from an envelope protein, preferably from HIV Env. As noted above, the envelope protein of HIV-1 is a glycoprotein of about 160 kd (gp160). During virus infection of the host cell, gp160 is cleaved by host cell proteases to form gp 120 and the integral membrane protein, gp41. The gp41 portion is anchored in (and spans) the membrane bilayer of virion, while the gp120 segment protrudes into the surrounding environment. As there is no covalent attachment between gp120 and gp41, free gp120 is released from the surface of virions and infected cells. Env polypeptides also include gp140 polypeptides, particularly o-gp140.

In certain embodiments, the Env polypeptide component of the composition is a monomer or a dimer. In preferred embodiments, the Env polypeptide component is an oligomeric (e.g., trimeric) Env polypeptide (e.g., o-gp140).

Furthermore, any of the Env glycoproteins described herein may also be liganded (e.g., complexed) to one or more molecules, including for example, CD4 and/or CD4 mimetics (see, e.g., U.S. Pat. No. 6,689,879; International Patent Publication WO 04/037847; Fouts et al. (2002) Proc. Natl. Acad. Sci. USA. 99(18):11842-11847), CCR5 co-receptors (Mkrtchyan et al. (2005) J. Virol. 79(17):11161-11169) or mimetics thereof, that (WO 2005/090391), polynucleotides (e.g., oligonucleotides such as CpGs, etc.), polypeptides (e.g., other viral proteins), small molecules, and combinations and/or other viral proteins.

The Env glycoproteins described herein can be derived one or more known HIV isolates, as well as newly identified isolates, and subtypes of these isolates. Thus, one of ordinary skill in the art in view of the teachings of the present disclosure and the art can determine corresponding regions in other HIV variants (e.g., isolates HIV_{IIIb}, HIV-1_{SF162}, HIV-1_{SF170}, HIV_{LAV}, HIV_{LAI}, HIV_{MN}, HIV-1_{CM235}, HIV-1_{US4}, other HV-1 strains from diverse subtypes, HIV-2 strains and diverse subtypes (e.g., HIV-2_{UC1} and HIV-2_{UC2}), and simian immunodeficiency virus (SIV). (See, e.g., Virology, 3rd Edition (W. K. Joklik ed. 1988); Fundamental Virology, 2nd Edition (B. N. Fields and D. M. Knipe, eds. 1991); Fields, B. N. et al. (eds.), Fields Virology, 4th edition (Lippincott Williams & Wilkins, 2001), for a description of these and other related viruses), using for example, sequence comparison programs (e.g., BLAST and others described herein) or identification and alignment of structural features (e.g., a program such as the “ALB” program described herein that can identify β-sheet regions). The actual amino acid sequences of the modified Env polypeptides can be based on any HIV variant.

The Env polypeptides described herein may include additional modifications to the native sequence, such as additional internal deletions, additions and substitutions. These modifications may be deliberate, as through site-directed mutagenesis, or may be accidental, such as through naturally occurring mutational events. Thus, for example, if the Env polypeptide is to be used in vaccine compositions, the modifications must be such that immunological activity (i.e., the ability to elicit an antibody response to the polypeptide) is not lost. Similarly, if the polypeptides are to be used for diagnostic purposes, such capability must be retained. The Env polypeptides described herein can be monomeric or oligomeric.

In preferred embodiments, Env glycoproteins from at least two different HIV subtypes are used. Based on phylogenetic analysis of HIV-1 nucleotide and amino acid sequences, HIV-1 isolates have been grouped into three groups, group M (major), group O (outlier) and group N (a new varient). Group M includes at least ten subtypes (designated A, (e.g., A1, A2), B, C, D, E, F (e.g., F1, F2), G, H, J and K) and CRFs. Variation in envelope amino acid sequences between different clades may exceed 30%. In addition, a significant proportion of HIV-specific neutralizing antibodies and CTL are type-specific. Accordingly, it is preferable that the compositions comprise multiple Env glycoproteins, for example, including HIV Env glycoproteins from subtypes A and B; B and C; A and C; A and E; B and E; C and E; A, B and C; A, B and E; A, C and E; B, C and E; A, B, C and E; A, B, C and F, etc.

In other embodiments, the Env glycoproteins from two or more (at least two) HIV Env polypeptides, wherein at least two of the Env polypeptides are each from different HIV types (e.g., HIV-1, HIV-2), are used. In still other embodiments, the Env glycoproteins from two or more (at least two) HIV Env polypeptides, wherein at least two of the Env polypeptides are each from different strains from the same subtypes (e.g., HIV-1_{SF2}, HIV-1_{SF162}, HIV-1_{CM235}, etc), are used.

Adjuvants

In a particular embodiment, the Env polypeptides described herein are adjuvanted, i.e., used in combination with one or more adjuvants or immunoregulatory agents. Adjuvants are substances that specifically or nonspecifically enhance the immune response to an antigen and include, for example, immunopotentiating molecules such as CpG oligos.

Examples of adjuvants that may be used in the compositions described herein include, but are not limited to, one or more of the following set forth below:

A. Oil-Emulsions

Oil-emulsion compositions and formulations suitable for use as adjuvants in the invention (with or without other specific immunostimulating agents such as muramyl peptides or bacterial cell wall components) include squalene-water emulsions, such as MF59 (5% Squalene, 0.5% Tween 80, and 0.5% Span 85, formulated into submicron particles using a microfluidizer). See WO 90/14837. See also, Podda (2001) Vaccine 19: 2673-2680; Frey et al. (2003) Vaccine 21:4234-4237. MF59 is used as the adjuvant in the FLUAD™ influenza virus trivalent subunit vaccine.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyethylenesorbitan monooleate), and/or 0.25-1.0% Span™ (sorbitan trioleate), and, optionally, N-acetylmuramyl-L-alanyl-D-isogluatminyl-L-alanine-2-(1′-2′-dipalmitoyl-sn-glycero-3-huydroxyphosphophoryloxy)-ethylamine (MTP-PE), for example, the submicron oil-in-water emulsion known as “MF59” (WO 90/14837; U.S. Pat. No. 6,299,884; U.S. Pat. No. 6,451,325; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) (New York: Plenum Press) 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in WO 90/14837; U.S. Pat. No. 6,299,884; and U.S. Pat. No. 6,451,325.

Complete Freund's adjuvant (CFA) and incomplete Freund's adjuvant (IFA) may also be used as adjuvants in the invention.

B. Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphosphates, orthophosphates), sulfates, etc. (see, e.g., Vaccine Design: The Subunit and Adjuvant Approach (Powell, M. F. and Newman, M. J. eds.) (New York: Plenum Press) 1995, Chapters 8 and 9), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO 00/23105).

Aluminum salts may be included in vaccines of the invention such that the dose of Al³⁺ is between 0.2 and 1.0 mg per dose.

In one embodiment the aluminum based adjuvant for use in the present invention is alum (aluminum potassium sulfate (AlK(SO₄)₂)), or an alum derivative, such as that formed in-situ by mixing an antigen in phosphate buffer with alum, followed by titration and precipitation with a base such as ammonium hydroxide or sodium hydroxide.

Another aluminum-based adjuvant for use in vaccine formulations of the present invention is aluminum hydroxide adjuvant (Al(OH)₃) or crystalline aluminum oxyhydroxide (AlOOH), which is an excellent adsorbant, having a surface area of approximately 500 m²/g. Alternatively, aluminum phosphate adjuvant (AlPO₄) or aluminum hydroxyphosphate, which contains phosphate groups in place of some or all of the hydroxyl groups of aluminum hydroxide adjuvant is provided. Preferred aluminum phosphate adjuvants provided herein are amorphous and soluble in acidic, basic and neutral media.

In another embodiment the adjuvant of the invention comprises both aluminum phosphate and aluminum hydroxide. In a more particular embodiment thereof, the adjuvant has a greater amount of aluminum phosphate than aluminum hydroxide, such as a ratio of 2:1, 3:1, 4:1, 5:1, 6:1, 7:1, 8:1, 9:1 or greater than 9:1, by weight aluminum phosphate to aluminum hydroxide. More particular still, aluminum salts in the vaccine are present at 0.4 to 1.0 mg per vaccine dose, or 0.4 to 0.8 mg per vaccine dose, or 0.5 to 0.7 mg per vaccine dose, or about 0.6 mg per vaccine dose.

Generally, the preferred aluminum-based adjuvant(s), or ratio of multiple aluminum-based adjuvants, such as aluminum phosphate to aluminum hydroxide is selected by optimization of electrostatic attraction between molecules such that the antigen carries an opposite charge as the adjuvant at the desired pH. For example, aluminum phosphate adjuvant (iep=4) adsorbs lysozyme, but not albumin at pH 7.4. Should albumin be the target, aluminum hydroxide adjuvant would be selected (iep 11.4). Alternatively, pretreatment of aluminum hydroxide with phosphate lowers its isoelectric point, making it a preferred adjuvant for more basic antigens.

C. Saponin Formulations

Saponin formulations are also suitable for use as adjuvants in the invention. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponins can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. Saponin adjuvant formulations include STIMULON® adjuvant (Antigenics, Inc., Lexington, Mass.).

Saponin compositions have been purified using High Performance Thin Layer Chromatography (HP-TLC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO 96/33739).

Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP 0 109 942, WO 96/11711 and WO 96/33739. Optionally, the ISCOMS may be devoid of (an) additional detergent(s). See WO 00/07621.

A review of the development of saponin based adjuvants can be found in Barr et al. (1998) Adv. Drug Del. Rev. 32:247-271. See also Sjolander et al. (1998) Adv. Drug Del. Rev. 32:321-338.

D. Virosomes and Virus Like Particles (VLPs)

Virosomes and Virus Like Particles (VLPs) are also suitable as adjuvants for use in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO 03/024480; WO 03/024481; Niikura et al. (2002) Virology 293:273-280; Lenz et al. (2001) J. Immunol. 166(9):5346-5355; Pinto et al. (2003) J. Infect. Dis. 188:327-338; and Gerber et al. (2001) J. Virol. 75(10):4752-4760. Virosomes are discussed further in, for example, Gluck et al. (2002) Vaccine 20:B 10-B16. Immunopotentiating reconstituted influenza virosomes (IRIV) are used as the subunit antigen delivery system in the intranasal trivalent INFLEXAL™ product (Mischler and Metcalfe (2002) Vaccine 20 Suppl 5:B17-B23) and the INFLUVAC PLUS™ product.

E. Bacterial or Microbial Derivatives

Adjuvants suitable for use in the invention include bacterial or microbial derivatives such as:

(1) Non-toxic derivatives of enterobacterial lipopolysaccharide (LPS): Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg. Med. Chem. Lett. 9:2273-2278.

(2) Lipid A Derivatives: Lipid A derivatives include derivatives of lipid A from Escherichia coli such as OM-174. OM-174 is described for example in Meraldi et al. (2003) Vaccine 21:2485-2491; and Pajak et al. (2003) Vaccine 21:836-842.

(3) Immunostimulatory oligonucleotides: Immunostimulatory oligonucleotides or polymeric molecules suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory. The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla et al. (2003) Nucl. Acids Res. 31(9): 2393-2400; WO 02/26757; and WO 99/62923 for examples of possible analog substitutions. The adjuvant effect of CpG oligonucleotides is further discussed in Krieg (2003) Nat. Med. 9(7):831-835; McCluskie et al. (2002) FEMS Immunol. Med. Microbiol. 32:179-185; WO 98/40100; U.S. Pat. No. 6,207,646; U.S. Pat. No. 6,239,116; and U.S. Pat. No. 6,429,199.

The CpG sequence may be directed to TLR9, such as the motif GTCGTT or TTCGTT. See Kandimalla et al. (2003) Biochem. Soc. Trans. 31 (part 3):654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell et al. (2003) J. Immunol. 170(8):4061-4068; Krieg (2002) TRENDS Immunol. 23(2): 64-65; and WO 01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla et al. (2003) BBRC 306:948-953; Kandimalla et al. (2003) Biochem. Soc. Trans. 31(part 3):664-658; Bhagat et al. (2003) BBRC 300:853-861; and WO03/035836.

Immunostimulatory oligonucleotides and polymeric molecules also include alternative polymer backbone structures such as, but not limited to, polyvinyl backbones (Pitha et al. (1970) Biochem. Biophys. Acta 204(1):39-48; Pitha et al. (1970) Biopolymers 9(8):965-977), and morpholino backbones (U.S. Pat. No. 5,142,047; U.S. Pat. No. 5,185,444). A variety of other charged and uncharged polynucleotide analogs are known in the art. Numerous backbone modifications are known in the art, including, but not limited to, uncharged linkages (e.g., methyl phosphonates, phosphotriesters, phosphoamidates, and carbamates) and charged linkages (e.g., phosphorothioates and phosphorodithioates).

(4) ADP-ribosylating toxins and detoxified derivatives thereof: Bacterial ADP-ribosylating toxins and detoxified derivatives thereof may be used as adjuvants in the invention. Preferably, the protein is derived from E. coli (i.e., E. coli heat labile enterotoxin “LT”), cholera (“CT”), or pertussis (“PT”). The use of detoxified ADP-ribosylating toxins as mucosal adjuvants is described in WO 95/17211 and as parenteral adjuvants in WO 98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references: Beignon et al. (2002) Infect. Immun. 70(6):3012-3019; Pizza et al. (2001) Vaccine 19:2534-2541; Pizza et al. (2000) Int. J. Med. Microbiol: 290(4-5):455-461; Scharton-Kersten et al. (2000) Infect. Immun. 68(9):5306-5313; Ryan et al. (1999) Infect. Immun. 67(12):6270-6280; Partidos et al. (1999) Immunol. Lett. 67(3):209-216; Peppoloni et al. (2003) Vaccines 2(2):285-293; and Pine et al. (2002) J. Control Release 85(1-3):263-270. Numerical reference for amino acid substitutions is preferably based on the alignments of the A and B subunits of ADP-ribosylating toxins set forth in Domenighini et al. (1995) Mol. Microbiol. 15(6):1165-1167.

F. Bioadhesives and Mucoadhesives

Bioadhesives and mucoadhesives may also be used as adjuvants in the invention. Suitable bioadhesives include esterified hyaluronic acid microspheres (Singh et al. (2001) J. Cont. Release 70:267-276) or mucoadhesives such as cross-linked derivatives of polyacrylic acid, polyvinyl alcohol, polyvinyl pyrollidone, polysaccharides and carboxymethylcellulose. Chitosan and derivatives thereof may also be used as adjuvants in the invention (see WO 99/27960).

G. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

H. Liposomes

Examples of liposome formulations suitable for use as adjuvants in the invention are described in U.S. Pat. No. 6,090,406; U.S. Pat. No. 5,916,588; and EP 0 626 169.

I. Polyoxyethylene Ether and Polyoxyethyethylene Ester Formulations

Adjuvants suitable for use in the invention include polyoxyethylene ethers and polyoxyethylene esters (see, e.g., WO 99/52549). Such formulations further include polyoxyethylene sorbitan ester surfactants in combination with an octoxynol (WO 01/21207) as well as polyoxyethylene alkyl ethers or ester surfactants in combination with at least one additional non-ionic surfactant such as an octoxynol (WO 01/21152).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

J. Polyphosphazene (PCPP)

PCPP formulations suitable for use as adjuvants in the invention are described, for example, in Andrianov et al. (1998) Biomaterials 19(1-3):109-115; and Payne et al. (1998) Adv. Drug Del. Rev. 31(3):185-196.

K. Muramyl Peptides

Examples of muramyl peptides suitable for use as adjuvants in the invention include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

L. Imidazoquinoline Compounds

Examples of imidazoquinoline compounds suitable for use as adjuvants in the invention include Imiquimod and its analogues, which are described further in Stanley (2002) Clin. Exp. Dermatol. 27(7):571-577; Jones (2003) Curr. Opin. Investig. Drugs 4(2):214-218; and U.S. Pat. Nos. 4,689,338; 5,389,640; 5,268,376; 4,929,624; 5,266,575; 5,352,784; 5,494,916; 5,482,936; 5,346,905; 5,395,937; 5,238,944; and 5,525,612.

M. Thiosemicarbozone Compounds

Examples of thiosemicarbazone compounds suitable for use as adjuvants in the invention, as well as methods of formulating, manufacturing, and screening for such compounds, include those described in WO 04/60308. The thiosemicarbazones are particularly effective in the stimulation of human peripheral blood mononuclear cells for the production of cytokines, such as TNF-.

N. Tryptanthrin Compounds

Examples of tryptanthrin compounds suitable for use as adjuvants in the invention, as well as methods of formulating, manufacturing, and screening for such compounds, include those described in WO 04/64759. The tryptanthrin compounds are particularly effective in the stimulation of human peripheral blood mononuclear cells for the production of cytokines, such as TNF-.

The invention may also comprise combinations of aspects of one or more of the adjuvants identified above. For example, the following adjuvant compositions may be used in the invention:

(1) a saponin and an oil-in-water emulsion (WO 99/11241);

(2) a saponin (e.g., QS21)+a non-toxic LPS derivative (e.g. 3dMPL) (see WO 94/00153);

(3) a saponin (e.g., QS21)+a non-toxic LPS derivative (e.g. 3dMPL)+a cholesterol;

(4) a saponin QS21)+3dMPL+IL-12 (optionally+a sterol) (WO 98/57659);

(5) combinations of 3dMPL with, for example, QS21 and/or oil-in-water emulsions (see EP 0 835 318; EP 0 735 898; and EP 0 761 231);

(6) SAF, containing 10% Squalane, 0.4% Tween 80, 5%pluronic-block polymer L121, and thr-MDP, either microfluidized into a submicron emulsion or vortexed to generate a larger particle size emulsion;

(7) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™);

(8) one or more mineral salts (such as an aluminum salt)+a non-toxic derivative of LPS (such as 3dPML);

(9) one or more mineral salts (such as an aluminum salt)+an immunostimulatory oligonucleotide (such as a nucleotide sequence including a CpG motif).

O. Human Immunomodulators

Human immunomodulators suitable for use as adjuvants in the invention include cytokines, such as interleukins (e.g. IL-2, IL-4, IL-5, IL-6, IL-7, IL-12, etc.), interferons (e.g. interferon-γ), macrophage colony stimulating factor (M-CSF), and tumor necrosis factor (TNF).

In certain embodiments, the compositions may be administered mucosally and will preferably further comprise a mucosal adjuvant. Suitable mucosal adjuvants include: CpG containing oligo, bioadhesive polymers, see WO 99/62546 and WO 00/50078; E. coli heat-labile entertoxin (“LT”) or detoxified mutants thereof or cholera toxin (“CT”) or detoxified mutant thereof or microparticles that are formed from materials that are biodegradeable and non-toxic, Preferred LT mutants include K63 or R72. See e.g., WO 93/13202, EP 0 620 850 B1, WO 97/02348, and WO 97/29771.

In a particularly preferred embodiment, the Env glycoproteins are adjuvanted with MF59, a CpG oligo (e.g., CpG-7909) or both MF59 and a CpG oligo. Although the precise mechanisms of adjuvant action for CpG-7909 and MF-59 are still subjects of intensive research, ample evidence suggests that CpG-7909/2006 activates B cells and increases production of costimulatory molecules in plasmacytoid dendritic cells (Kerkmann et al. (2003) J. Immunol. 170(9):4465-4474), while MF59 interacts with antigen presenting cells and is internalized by dendritic cells at the site of an intramuscular injection (Dupois (1998) Cell. Immunol. 186(1):18-27). Both CpG-7909 and MF59 are licensed for human use and have been well tolerated in clinical trials (Kahn et al. (1994) J. Infect. Dis. 70(5):1288-1291; Ott et al. (1995) Pharm. Biotechnol. 6:277-296; Cooper et al. (2004) Vaccine 22(23-24):3136-3143), including hepatitis-B vaccine and influenza vaccine trials performed in people living with HIV (Cooper et al. (2005) AIDS 19(14):1473-1479; Gabutti et al. (2005) J. Int. Med. Res. 33(4):406-416). Experiments described herein demonstrate the ability of MF59 and CpG-7909 to enhance the quality and breadth of immune responses elicited by multivalent HIV o-gp140.

Polypeptide Production

The Env polypeptides of the present invention can be produced in any number of ways known in the art.

In one embodiment, the polypeptides are generated using recombinant techniques, well known in the art. In this regard, oligonucleotide probes can be devised based on the known sequences of the Env (e.g., gp140) polypeptide genome and used to probe genomic or cDNA libraries for Env genes. The gene can then be further isolated using standard techniques and, e.g., restriction enzymes employed to truncate the gene at desired portions of the full-length sequence. Similarly, the Env gene(s) can be isolated directly from cells and tissues containing the same, using known techniques, such as phenol extraction and the sequence further manipulated to produce any desired truncations. See, e.g., Sambrook et al., supra, for a description of techniques used to obtain and isolate DNA.

The genes encoding the Env glycoproteins can be produced synthetically, based on the known sequences. The nucleotide se_{q}uence can be designed with the appropriate codons for the particular amino acid sequence desired. The complete sequence is generally assembled from overlapping oligonucleotides prepared by standard methods and assembled into a complete coding sequence. See, e.g., Edge et al. (1981) Nature 292:756-762; Nambair et al. (1984) Science 223:1299-1301; Jay et al. (1984) J. Biol. Chem. 259:6311-6317; Stemmer et al. (1995) Gene.164:49-53.

Recombinant techniques are readily used to clone a gene encoding an Env polypeptide gene that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al. (1990) PCR Applications: Protocols for Functional Genomics; Zoller and Smith (1983) Methods Enzymol. 100:468-500. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. (1982) Proc. Natl. Acad. Sci USA 79:6409-6413.

Once coding sequences for the desired Env glycoproteins have been isolated or synthesized, they can be cloned into any suitable vector or replicon for expression. Numerous cloning vectors are known to those of skill in the art, and the selection of an appropriate cloning vector is a matter of choice. Examples of recombinant DNA vectors for cloning and host cells which they can transform include the bacteriophage X (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), PIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith (1987) Texas Agricultural Experiment Station Bulletin No. 1555. Materials and methods for baculovirus/insect cell expression systems are commercially available in kit form from, inter alfa, Invitrogen, San Diego Calif. (“MaxBac” kit).

Plant expression systems can also be used to produce the modified Env proteins. Generally, such systems use virus-based vectors to transfect plant cells with heterologous genes. For a description of such systems see, e.g., Porta et al. (1996) Mol. Biotech. 5:209-221; and Hackland et al. (1994) Arch. Virol. 139:1-22.

Viral systems, such as a vaccinia based infection/transfection system, as described in Tomei et al. (1993) J. Virol. 67:4017-4026 and Selby et al. (1993) J. Gen. Virol. 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides or heterologous sequences can be used. Leader sequences can be removed by the host in post-translational processing. See, e.g., U.S. Pat. Nos. 4,431,739; 4,425,437; and 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector which already contains the control sequences and an appropriate restriction site.

In some cases it may be necessary to modify the coding sequence so that it may be attached to the control sequences with the appropriate orientation; i.e., ₜo maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transfect an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. In one preferred embodiment, o-gp 140 (trimeric) is produced and/or purified from CHO cells. See, Srivastava et al. (2002) J. Virol. 76(6):2835-2847; Srivastava et al. (2003) J. Virol. 77(29):11244-11259.

Bacterial hosts such as E. coli, Bacillus subtilis, and Streptococcus spp., may also find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerinzondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

_{—} Depending on the expression system and host selected, the proteins of the present invention are produced by growing host cells transformed by an expression vector described above under conditions whereby the protein of interest is expressed. The selection of the appropriate growth conditions is within the skill of the art.

In one embodiment, the transformed cells secrete the polypeptide product into the surrounding media. Certain regulatory sequences can be included in the vector to enhance secretion of the protein product, for example using a tissue plasminogen activator (TPA) leader sequence, an interferon (β or γ) signal sequence or other signal peptide sequences from known secretory proteins. The secreted polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the Env polypeptides substantially intact. Intracellular proteins can also be obtained by removing components from the cell wall or membrane, e.g., by the use of detergents or organic solvents, such that leakage of the Env polypeptides occurs. Such methods are known to those of skill in the art and are described in, e.g., Protein Purification Applications: A Practical Approach (E. L. V. Harris and S. Angal (eds.)) 1990.

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced Env polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular Env polypeptides of the present invention involves affinity purification, such as by immunoaffmity chromatography using anti-Env specific antibodies, or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the polypeptides can be further purified using conventional techniques well known in the art, such as by any of the techniques ‘described above.

Relatively small polypeptides, i.e., up to about 50 amino acids in length, can be conveniently synthesized chemically, for example by any of several techniques that are known to those skilled in the peptide art. In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer (eds.), The Peptides: Analysis, Synthesis, Biology, Vol. 1, for classical solution synthesis.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

The polypeptide analogs of the present invention can also be chemically prepared by other methods such as by the method of simultaneous multiple peptide synthesis. See, e.g., Houghten (1985) Proc. Natl. Acad. Sci. USA 82:5131-5135; U.S. Pat. No. 4,631,211.

Antibodies

Antibodies, both monoclonal and polyclonal, which are directed against adjuvanted HIV glycoproteins as described herein may find use in diagnosis and therapeutic applications, for example, those antibodies which are neutralizing are useful in passive immunotherapy. Monoclonal antibodies, in particular, may be used to raise anti-idiotype antibodies.

Anti-idiotype antibodies are immunoglobulins which carry an “internal image” of the antigen of the infectious agent against which protection is desired_{—} Techniques for raising anti-idiotype antibodies are known in the art. See, e.g., Grzych et al. (1985) Nature 316:74-76; MacNamara et al. (1984) Science 226:1325-1326, Uytdehaag et al (1985) J. Immunol. 134:1225-1229. These anti-idiotype antibodies may also be useful for treatment and/or diagnosis of HIV.

An immunoassay for viral antigen may use, for example, a monoclonal antibody directed towards a viral epitope, a combination of monoclonal antibodies directed towards epitopes of one viral polypeptide, monoclonal antibodies directed towards epitopes of different viral polypeptides, polyclonal antibodies directed towards the same viral antigen, polyclonal antibodies directed towards different viral antigens or a combination of monoclonal and polyclonal antibodies.

Immunoassay protocols may be based, for example, upon competition, or direct reaction, or sandwich type assays. Protocols may also, for example, use solid supports, or may be by immunoprecipitation. Most assays involve the use of labeled antibody or polypeptide. The labels may be, for example, fluorescent, chemiluminescent, radioactive, or dye molecules. Assays which amplify the signals from the probe are also known. Examples of which are assays which utilize biotin and avidin, and enzyme-labeled and mediated immunoassays, such as ELISA assays.

An enzyme-linked immunosorbent assay (ELISA) can be used to measure either antigen or antibody concentrations. This method depends upon conjugation of an enzyme to either an antigen or an antibody, and uses the bound enzyme activity as a quantitative label. To measure antibody, the known antigen is fixed to a solid phase (e.g., a microplate or plastic cup), incubated with test serum dilutions, washed, incubated with anti-immunoglobulin labeled with an enzyme, and washed again. Enzymes suitable for labeling are known in the art, and include, for example, horseradish peroxidase. Enzyme activity bound to the solid phase is measured by adding the specific substrate, and determining product formation or substrate utilization colorimetrically. The enzyme activity bound is a direct function of the amount of antibody bound.

To measure antigen, a known specific antibody is fixed to the solid phase, the test material containing antigen is added, after an incubation the solid phase is washed, and a second enzyme-labeled antibody is added. After washing, substrate is added, and enzyme activity is estimated colorimetrically, and related to antigen concentration.

Polyclonal antibodies can be produced by administering the fusion protein to a mammal, such as a mouse, a rabbit, a goat or a horse. Serum from the immunized animal is collected and the antibodies are purified from the plasma by, for example, precipitation with ammonium sulfate, followed by chromatography, preferably affinity chromatography. Techniques for producing and processing polyclonal antisera are known in the art.

Monoclonal antibodies can also be produced. Normal B cells from a mammal, such as a mouse, immunized with, e.g., a mutant NS3 polypeptide or NS-core fusion protein can be fused with, for example, HAT-sensitive mouse myeloma cells to produce hybridomas. Hybridomas can be identified using RIA or ELISA and isolated by cloning in semi-solid agar or by limiting dilution. Clones producing the desired specific antibodies are isolated by another round of screening.

Antibodies, monoclonal and polyclonal, which are directed against epitopes, are particularly useful for detecting the presence of antigens in a sample, such as a serum sample from an HIV-infected human. An immunoassay for an HIV antigen may utilize one antibody or several antibodies. An immunoassay for an HIV antigen may use, for example, a monoclonal antibody directed towards an HIV epitope, a combination of monoclonal antibodies directed towards epitopes of one Env, monoclonal antibodies directed towards epitopes of different polypeptides, polyclonal antibodies directed towards the same HIV antigen, polyclonal antibodies directed towards different HIV antigens, or a combination of monoclonal and polyclonal antibodies. Immunoassay protocols may be based, for example, upon competition, direct reaction, or sandwich type assays using, for example, labeled antibody. The labels may be, for example, fluorescent, chemiluminescent, or radioactive.

The polyclonal and monoclonal antibodies may further be used to isolate Env by immunoaffinity columns. The antibodies can be affixed to a solid support by, for example, adsorption or by covalent linkage so that the antibodies retain their immunoselective activity. Optionally, spacer groups may be included so that the antigen binding site of the antibody remains accessible. The immobilized antibodies can then be used to bind the target from a biological sample, such as blood or plasma. The bound proteins or complexes are recovered from the column matrix by, for example, a change in pH.

Diagnostic, Vaccine and Therapeutic Applications

The compositions of the present invention or the polynucleotides coding therefor, can be used for a number of diagnostic and therapeutic purposes. For example, the proteins and polynucleotides or antibodies generated against the same, can be used in a variety of assays, to determine the presence of reactive antibodies/and or Env proteins in a biological sample to aid in the diagnosis of HIV infection or disease status or as measure of response to immunization.

As noted above, the presence of antibodies reactive with the Env (e.g., o-gp140) polypeptides and, conversely, antigens reactive with antibodies generated thereto, can be detected using standard electrophoretic and immunodiagnostic techniques, including immunoassays such as competition, direct reaction, or sandwich type assays. Such assays include, but are not limited to, western blots; agglutination tests; enzyme-labeled and mediated immunoassays, such as ELISAs; biotin/avidin type assays; radioimmunoassays; immunoelectrophoresis;.immunoprecipitation, etc.

The reactions generally include revealing labels such as fluorescent, chemiluminescent, radioactive, or enzymatic labels or dye Molecules, or other methods for detecting the formation of a complex between the antigen and the antibody or antibodies reacted therewith.

Solid supports can be used in the assays such as nitrocellulose, in membrane or microtiter well form; polyvinylchloride, in sheets or microtiter wells; polystyrene latex, in beads or microtiter plates; polyvinylidine fluoride; diazotized paper; nylon membranes; activated beads, and the like.

The adjuvanted Env glycoprotein compositions described herein, or antibodies to the compositions, can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits.

The compositions may also be used as vaccines to induce a prophylactic (i.e., to prevent infection and/or disease progression) and/or a therapeutic (to treat HIV following infection) immune response in a subject. The vaccine compositions can comprise mixtures of Env glycoproteins (or nucleotide sequences encoding the proteins) derived from more than one viral isolate and/or subtype. In certain embodiments, the compositions can comprise mixtures of Env glycoproteins (or nucleotide sequences encoding the proteins) derived from at least two different subtypes (e.g., subtype B and C). In other embodiments, the compositions can comprise mixtures of Env glycoproteins (or nucleotide sequences encoding the proteins) derived from at least three different subtypes (e.g., subtypes A, B and C). In further embodiments, the compositions can comprise mixtures of Env glycoproteins (or nucleotide sequences encoding the proteins) derived from at least two different REV types (e.g., HIV-1, HIV-2). In still other embodiments, the compositions can comprise mixtures of Env glycoproteins (or nucleotide sequences encoding the proteins) derived from at least two different strains from the same subtypes (e.g., HIV-1_{SF2}, HIV-1_{SF162}, HIV-1_{CM235}, etc).

In particular embodiments, the multivalent compositions described herein can be used to induce an immune response which protects against and/or treats infection from multiple HIV types, strains and/or subtypes. In other embodiments, a multivalent composition described herein can be used to induce a prophylactic and/or therapeutic immune response against HIV strains from multiple HIV subtypes. For example, a multivalent composition described herein can be used to induce a prophylactic and/or, therapeutic immune response against HIV subtypes that include the strains from which the HIV Env glycoproteins of the compositions are derived. In a particular embodiment, a multivalent composition described herein can be used to induce a prophylactic and/or therapeutic immune response against HIV subtypes that include the strains from which the HIV Env glycoproteins of the compositions are derived and against HIV subtypes that are not represented in the multivalent composition.

The compositions and vaccines described herein may produce broad neutralizing activity against a variety of subtypes, including subtypes that do not form part of the immunization composition. For example, in Example 1, Applicants have demonstrated that immunization with a multivalent composition including subtype B and C Env glycoproteins elicited neutralizing antibodies against a variety of subtype B, C and A HIV strains.

The compositions described herein may also be administered in conjunction with other antigens and immunoregulatory agents, for example, immunoglobulins, cytokines, lymphokines, and chemokines, including but not limited to IL-2, modified IL-2 (cys125-ser125), GM-CSF, IL-12, γ-interferon, IP-10, MIP1 and RANTES.

The compositions may be administered as polypeptides or as polynucleotides encoding the polypeptides. Polynucleotides may be delivered as naked nucleic acid vaccines (e.g., DNA) or using viral vectors such as retroviral vectors, adenoviral vectors, alphavirus vectors (see, e.g., U.S. Pat. Nos. 6,465,634; 6,458,560; 6,451,592; 6,426,196; 6,376,236; 6,015,694; 6,342,372; 6,015,686; 5,843,723; and 5,789,245) and adeno-associated viral vectors. Polynucleotides may also be delivered using non-viral vectors (e.g., liposomes, particles coated with nucleic acid or protein). These and other polynucleotide delivery systems are known to those of skill in the art and are described, for example, in U.S. Pat. Nos. 6,943,153; 6,602,705; and U.S. Patent Publication Nos. 20050214256; 20030194800; 20030170614; 20030223964; 20030143248; and 20020198621.

The compositions may also comprise a mixture of protein and nucleic acid, which in turn may be delivered using the same or different vehicles.

The compositions and vaccines may be administered in a single or multiple modalities (e.g., a DNA or viral prime and a protein boost), and the separate modalities may be administered sequentially or concomitantly. For example, in certain embodiments, a composition described herein may be administered to prime a mammalian subject. Priming, as used herein, means any method whereby a first immunization with a composition described herein permits the generation of an immune response to a target antigen or antigens upon a second immunization with a second composition described herein, wherein the second immune response is greater than that achieved where the first immunization is either not provided or where the first immunization administered contains composition which does not express the antigen or antigens. Priming encompasses regimens which include a single dose or multiple dosages, administered hourly, daily, weekly, monthly or yearly. In a particular embodiment, priming (or priming immunization) comprises at least two administrations (comprising one or more dose or dosage). For example, in a particular embodiment, priming by administration of one or more compositions described herein entails at least one (e.g., 1, 2, 3, 4, 5, 6, 7 or more) administration(s) (comprising one or more dose or dosage) of the composition(s). The time interval between administrations can be hours, days, weeks, months or years. In other embodiments, a composition described herein may be administered as a booster to boost the immune response achieved after priming of the mammalian subject. Compositions administered as a booster are administered some time afterpriming. In a particular embodiment, boosting (or boosting immunization) may be about two (2) to twenty-seven (27) weeks after priming (or priming immunization). Boosting encompasses regimens which include a single dose or multiple dosages, administered hourly, daily, weekly, monthly or yearly. In certain embodiments, boosting (or boosting immunization) comprises at least one administration. In other embodiments, boosting (or boosting immunization) comprises at least two administrations (comprising one or more dose or dosage). For example, in such instance, in a particular embodiment, boosting by administration of one or more compositions described herein entails at least one (e.g., 1, 2, 3, 4, 5, 6, 7 or more) administrations (comprising one or more dose or dosage) of the composition(s). The time interval between administrations can be hours, days, weeks, months or years.

In certain embodiments, the same composition can be administered as the prime and as the booster. In other embodiments, different compositions can be used for priming and for boosting. For example, in certain embodiments, multiple immunizations of polypeptide compositions are administered as primes and/or boosts. In other embodiments, one or more polynucleotide (e.g., plasmid, alphavirus vector, poxvirus vector, adenovirus vector, or combinations thereof) priming immunizations are administered followed by one or more polypeptide boosts.

The vaccines described herein generally include one or more “pharmaceutically acceptable excipients or vehicles” such as water, saline, glycerol, ethanol, etc. Additionally, auxiliary substances, such as wetting or emulsifying agents, pH buffering substances, and the like, may be present in such vehicles.

A carrier is optionally present which is a molecule that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the Env polypeptide may be conjugated to a bacterial toxoid, such as toxoid from diphtheria, tetanus, cholera, etc.

Typically, the vaccine compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above.

The vaccines described herein comprise a therapeutically effective amount of an adjuvanted HIV Env glycoprotein composition, or nucleotide sequences encoding the same, antibodies directed to these complexes and any other of the above-mentioned components, as needed. By “therapeutically effective amount” is meant an amount that induces a protective immunological response in the uninfected, infected or unexposed individual to whom the vaccine is administered. Such a response will generally result in the development in the subject of a secretory, cellular and/or antibody-mediated immune response to the vaccine. Cellular-mediated immune responses include CD4+ T helper cell responses, cytotoxic T lymphocytes, CD8+ cell antiviral responses and antiviral chemokine responses. Antibody-mediated immune responses include those measured by serologic assays (such as virus neutralization assays, assays for ADCC, ELISAs, immunoblot assays). Thus, a protective immunological response includes, but is not limited to, one or more of the following effects: the production of antibodies from any of the immunological classes, such as immunoglobulins A, D, E, G or M; the proliferation of B and T lymphocytes; the provision of activation, growth and differentiation signals to immunological cells; expansion of helper T cell, suppressor T cell and/or cytotoxic T cell.

Preferably, the effective amount is sufficient to bring about treatment or prevention of disease symptoms. The exact amount necessary will vary depending on the subject being treated; the age and general condition of the individual to be treated; the capacity of the individual's immune system to synthesize antibodies; the degree of protection desired; the severity of the condition being treated; the particular multivalent composition selected and its mode of administration, among other factors. An appropriate effective amount can be readily determined by one of skill in the art. A “therapeutically effective amount” will fall in a relatively broad range that can be determined through routine trials.

Both nucleic acids and/or polypeptides can be injected either subcutaneously, epidermally, intradermally, intramucosally such as nasally, rectally and vaginally, intraperitoneally, intravenously, orally or intramuscularly. Other modes of administration include oral and pulmonary administration, suppositories, needle-less injection, transcutaneous and transdermal applications. Dosage treatment may be a single dose schedule or a multiple dose schedule. Administration of nucleic acids may be combined with administration of peptides or other substances.

While the invention has been described in conjunction with the preferred specific embodiments thereof, it is to be understood that the foregoing description as well as the examples which follow are intended to illustrate and not limit the scope of the invention. Other aspects, advantages and modifications within the scope of the invention will be apparent to those skilled in the art to which the invention pertains.

### EXAMPLES

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example

### Immunization with Multivalent O-GP140 Polypeptides

In an attempt to enhance the immunogenicity of the HIV env, we and others have been pursuing an oligomeric HIV env glycoprotein (o-gp140) subunit vaccine approach based on the design of HIV env antigens that may mimic the native, trimeric env spikes present on the surface of the HIV virion. See, e.g., Yang et al. (2000) J. Virol. 74(12):5716-5725; Grundner et al. (2005) Virology 331(1):33-46; Srivastava et al. (2003) J. Virol. 77(29):11244-11259; Barnett et al. (2001) J. Virol. 75(12):5526-5540. We have thus developed soluble HIV o-gp140s which have been modified in the second hypervariable loop (ΔV2) and which have been derived from the North American, clade-B HIV strain SF162 and more recently, from the South African, clade-C HIV strain TV1 (Lian et al. (2005) J. Virol. 79(21):13338-13349).

Using prime/boost vaccine regimens involving DNA or adenovirus priming immunizations followed by oligomeric protein immunizations in MF59 adjuvant, we have shown that these ΔV2 o-gp140's are immunogenic in rabbits (Barnett et al. (2001) J. Virol. 75(12):5526-5540), rhesus macaques (Otten et al. (2005) J. Virol. 79(13):8189-8200) and chimpanzees (Peng et al. (2005) J. Virol. 79(16):10200-10209). Although promising signs of broadly-neutralizing antibody activity when priming chimpanzees with replicating adenovirus-HIV recombinants, followed by boosting with SF162 ΔV2 o-gp140 (Peng et al. (2005) J. Virol. 79(16):10200-10209), the breadth of the humor response against heterologous HIV strains has been limited when using DNA prime/o-gp140 boost vaccine regimens in rabbits and rhesus monkeys (Lian et al. (2005) J. Virol. 79(21):13338-13349). Nonetheless, when combined in a DNA prime/protein boost regimen, the ΔV2 TV1 and SF162 gp140 immunogens have consistently yielded the highest titer of neutralizing antibodies against the neutralization-sensitive SF162 strain in several studies (Lian et al. (2005) J. Virol. 79(21):13338-13349), suggesting that the combination of these gp140 antigens into a single bivalent vaccine could be promising, at least in terms of potency.

In agreement with recent evidence indicating that polyvalent and multiclade HIV vaccines elicit improved signs of breadth of neutralizing antibody responses in rhesus macaques (Seaman et al. (2005) J. Virol. 79(5):2956-2963; Pal et al. (2005) J. Med. Primatol. 34(5-6):226-236) and in guinea pigs (Chakrabarty et al. (2005) Vaccine. 23(26):3434-3445), the following protein-only, bivalent ΔV2 o-gp140 experiments were performed to assess the breadth of neutralizing antibody responses against a panel of well-characterized HIV pseudotyped strains derived from acute/early HIV infections. Isolates selected were those derived early during the course of HIV infection, on the grounds that early isolates would better represent HIV strains that are transmitted and would thus be more relevant in an HIV vaccine setting (Moore et al. (2004) Nat. Med. 10(8):769-771). In addition, the subtype-B panel was chosen to maintain vaccine immune monitoring consistency, based on recent recommendations for the global assessment of HIV neutralizing antibodies (Li et al. (2005) J. Virol. 79(16):10108-10125; Mascola et al. (2005) J. Virol. 79(16):10103-10107; Esparza (2005) Int. Microbial. 8(2):93-101).

A. Rabbits

Animals, Vaccines and adjuvants

Research-grade SF162 ΔV2 env o-gp140 and TV1 ΔV2 env o-gp140 were prepared as described previously in Srivastava et al. (2003) J. Virol. 77(20):11244-11259 and Lian et al. (2005) J. Virol. 79(21):13338-13349. Ten New Zealand White rabbits were used per immunization group. Rabbits received a total of four intramuscular immunizations in the gluteus on weeks 0, 4, 12 and 24. Vaccine doses were 25 μg of the indicated o-gp140 vaccines per animal. Animals in the bivalent vaccine groups (groups 5 & 6) received 12.5 μg of SF162 o-gp140 and 12.5 μg of TVI o-gp140 combined into the same syringe. Animals in groups 1, 3 and 5 received protein immunizations combined with 250 μl of MF59 adjuvant; animals in groups 2, 4 and 6 received protein immunizations combined with 250 μl MF59 adjuvant and 500 μg of CpG-7909.

Rabbits were immunized with monovalent or bivalent ΔV2 o-gp140 vaccines in MF59 adjuvant, with or without the immunopotentiator CpG-7909 as shown in the following Tables:

Section A

| | | | | | Total | Vol/ | Sites/ | |
|---|---|---|---|---|---|---|---|---|
| Group | Animal # | Imm'n # | Adjuvant | Immunogen | Dose | Site | Animal | Route |
| 1 | 1-10 | 1, 2, 3, 4 | MF59C | o-gp140 dV2 SF162 | 25 μg | 0.25 ml | 2 | IM/Glut |
| | | | | | | | | (Needle) |
| 2 | 11-20 | 1, 2, 3, 4 | MF59C + | o-gp140 dV2 SF162 | 25 μg | 0.25 ml | 2 | IM/Glut |
| | | | CpG | | | | | (Needle) |
| 3 | 21-30 | 1, 2, 3, 4 | MF59C | o-gp140 dV2 TV1 | 25 μg | 0.25 ml | 2 | IM/Glut |
| | | | | | | | | (Needle) |
| 4 | 31-40 | 1, 2, 3, 4 | MF59C + | o-gp140 dV2 TV1 | 25 μg | 0.25 ml | 2 | IM/Glut |
| | | | CpG | | | | | (Needle) |
| 5 | 41-50 | 1, 2, 3, 4 | MF59C | o-gp140 dV2 SF162 | 25 μg | 0.25 m | 2 | IM/Glut |
| | | | | o-gp140 dV2 TV1 | (12.5 | | | (Needle) |
| | | | | | μg ea.) | | | |
| 6 | 51-60 | 1, 2, 3, 4 | MF59C + | o-gp140 dV2 SF162 | 25 μg | 0.25 ml | 2 | IM/Glut |
| | | | CpG | o-gp140 dV2 TV1 | (12.5 | | | (Needle) |
| | | | | | μg ea.) | | | |

| Adjuvant/Buffer | Reference | Description |
|---|---|---|
| MF59.C | Lot # 189011 | Microfluidized emulsion containing 5% squalene, 0.5% Tween |
| | Part | 80, 0.5% span 85, in 10 mM citrate pH 6. |
| | OKBZO15180 | 10 mL aliquots, store at 4° C. |
| | Exp 27 Aug. 2006 | |
| CpG 7909 | Lot # 207-03- | 7909 CpG ODN procured from Coley Pharm (10 mg/ml). Group |
| | 002 | and administered with MF59C. Store at 2-8° C. |
| | Immu. 1: Vial | |
| | #4524 and part | |
| | of 4525. | |

| Antigen | Reference | Description |
|---|---|---|
| o-gp140 dV2 | Ref # | The oligomer protein contains five amino acid mutations in |
| SF162 protein | Lot# EK04NOV04 | the cleavage site in addition to the deletion of V2 region. |
| (research) | NB# 16991/112 | Protein was purified from CHO supernatant using a |
| | | combination of GNA, DEAE and CHAP columns. At the final |
| | | stage, the trimers are separated from the monomers using |
| | | gel filtration column. Purified protein is stored at −80° C. until |
| | | use. |
| o-gp140 dV2 TV1 | Ref # | The subtype C oligomer protein contains five amino acid |
| protein | Lot#EK20MAY04 | mutations in the cleavage site in addition to the deletion of |
| (research) | NB# 16991/112 | V2 region. Protein was purified from CHO expression. |

| Group | Preparation |
|---|---|
| 1, 3, 5 | Immunization 1-4: Protein Immunization + MIF59 |
| | Protein doses are 25 μg protein per animal. The initial protein is diluted to 0.100 mg/ml in |
| | PBS in a volume of 2.75 ml (containing 275 μg protein). Store at −80° C. until use. Thaw at |
| | room temperature; material should be clear with no particulate matter. Add equal volume of |
| | adjuvant to thawed protein and mix well by inverting the tube. Immunize each rabbit with |
| | 0.25 ml adjuvanted protein per side, IM/Glut for a total of 0.5 ml per animal. Use material |
| | within 1 hour of the addition of adjuvant. Needles are used for injections. |
| 2, 4, 6 | Immunization 1-4: Protein Immunization + MF59 + CpG |
| | Protein doses are 25 ug protein per animal. The initial protein is diluted in PBS (275 μg in a |
| | volume of 2.2 ml). Store at −80° C. until use. Thaw at room temperature; material should be |
| | clear with no particulate matter. Add 550 μl CpG (based on 500 μg per animal, 50 μl from |
| | the 10 mg/ml stock) and 2.75 ml MF59 to thawed protein and mix well by inverting the tube. |
| | Immunize each rabbit with 0.25 ml adjuvanted protein per side, IM/Glut for a total of 1 ml per |
| | animal. Use material within 1 hour of the addition of adjuvant. Needles are used for |
| | injections. |

| Bleed: | 0 | 1 | 2 | 3 | 4 | 5 |
|---|---|---|---|---|---|---|
| Week: | 0 | 4 | 6 | 8 | 12 | 14 |
| Sample: | Clotted Bld. | Clotted Bld. | Clotted Bld. | Clotted Bld. | Clotted Bld. | Clotted Bld. |
| | for Serum | for Serum | for Serum | for Serum | for Serum | for Serum |
| Volume: | 20 cc each | 5 cc each | 20 cc each | 20 cc each | 20 cc each | 20 cc each |
| Method: | AA/MEV | AA/MEV | AA/MEV | AA/MEV | AA/MEV | AA/MEV |

| Bleed: | 6 | 7 | 8 | 9 | 10 through 17 |
|---|---|---|---|---|---|
| Week: | 16 | 24 | 26 | 28 | TBD |
| Sample: | Clotted Bld. | Clotted Bld. | Clotted Bld. | Clotted Bld. | Clotted Bld. |
| | for Serum | for Serum | for Serum | for Serum | for Serum |
| Volume: | 20 cc each | 20 cc each | 20 cc each | 20 cc each | 20 cc each |
| Method: | AA/MEV | AA/MEV | AA/MEV | AA/MEV | AA/MEV |

TZM-bl Screening Assay

Neutralizing antibody activity was measured as reductions in luciferase gene expression after a single round of virus infection in TZM-bl cells, as described previously Li et al. (2005) J. Virol. 79(16):10108-10255 and Montefiori (2004) (John Wiley & Sons, New York, N.Y.). Serum samples obtained before (pre-bleed), 2 weeks post-third (2wp3) and 2 weeks post-fourth (2wp4) immunizations were screened for neutralizing activity in parallel at a 1:10 dilution in duplicate. The percent reduction in relative luminescence units (RLU) was calculated relative to the RLU in the presence of pre-bleed serum. Similarly, neutralizing titers were determined by monitoring activity of sequentially diluted sera over a range from 1:15 to 1:32805.

The bivalent o-gp140 vaccines described here were shown to be superior to their monovalent vaccine counterparts in terms of potency against the clade B SF162, particularly when formulated in MF59 adjuvant with CpG-7909 and when the animals are immunized three or four times.

Additional experiments using HIV Env polypeptides derived from Thai subtype E strain HIV-1_{CM235} in addition to (or instead of) TV1 may be conducted. The results from these experiments are reasonably anticipated to similarly show an enhanced immune response as compared to monovalent vaccine compositions.

B. Macaques

Non-human primates (macaques) are also immunized with multivalent o-gp140 vaccines adjuvanted described above with MF-59 and CpG-7909 essentially as described below:

| GROUP | EXPERIMENTAL TREATMENT | BIOTECHNIQUES | NUMBER | SEX |
|---|---|---|---|---|
| 1 | 3x recombinant subtype A glycoprotein | IM Immunization, | 5 | m/f |
| | “461” gp140, in MF59 + CpG adjuvant, | IV challenge with | | |
| | 3 x SF162 gp140, (subtype B) in MF59 + | SHIV | | |
| | CpG and 3 x TV1 gp140 (subtype C) | Blood collection | | |
| | and/or CM235 gp140 (subtype E) in | | | |
| | MF59 + CpG. | | | |
| 4 | Controls injected with MF59 + CpG | IM injection | 5 | m/f |
| | adjuvant alone. | IV challenge with | | |
| | | SHIV | | |
| | | Blood collection | | |

The first immunization (week 0) is as follows: Groups 1: 3×50 μg of each gp140 Env protein antigen (subtype A, B, C) mixed in 1 ml of MF59+CpG IM (left upper arm); Group 4: 1 ml of MF59+CpG (in left upper arm). The second immunization (week 6) is as follows: Group 1: 3×50 μg of each protein antigen in 1 ml of MF59+CpG (in left upper arm); Group 4: 1 ml of MF59+CpG (in left upper arm). The third immunization (week 16) is as follows: Group 1: 3×50 μg of protein antigen in 1 ml of MF59+CpG (in left upper arm); Group 4: 1 ml of MF59+CpG (in left upper arm). The fourth (optional) immunization (week 28) is as follows: Group 1: 3×50 μg of protein antigen in 1 ml of MF59+CpG (in left upper arm); Group 4: 1 ml of MF59+CpG (in left upper arm).

The results herein demonstrate adjuvantation of a bivalent HIV env glycoprotein vaccine with MF59 and CpG-7909, alone or in combination, enhanced the potency of neutralizing antibody responses.

What is claimed is:

1. An immunogenic composition comprising a first HIV envelope polypeptide and a second HIV envelope polypeptide, wherein the first and second envelope polypeptides are from different HIV subtypes.

2. An immunogenic composition of claim 1, further comprising one or more adjuvants.

3. An immunogenic composition of claim 1 or claim 2, wherein the one or more adjuvants are selected from the group consisting of MF59, CpG molecules, microparticles, alum and combinations thereof.

4. An immunogenic composition of any one of the preceding claims, wherein the HIV envelope polypeptides comprise a polypeptide selected from the group consisting of: a gp120, a gp140 and a gp160 polypeptide.

5. The immunogenic composition of claim 4, wherein at least one HIV Env polypeptide comprises a gp140 polypeptide and said gp140 polypeptide is an oligomeric gp140 (o-gp140).

6. An immunogenic composition comprising two or more HIV envelope polypeptides, wherein at least two of the envelope polypeptides are each from different HIV subtypes.

7. The immunogenic composition of claim 6, comprising three or more HIV envelope proteins, wherein at least three of the envelope polypeptides are each from different HIV subtypes.

8. The immunogenic composition of claim 6 or claim 7, further comprising one or more adjuvants.

9. The immunogenic composition of claim 8, wherein the one or more adjuvants are selected from the group consisting of MF59, CpG molecules, microparticles, alum and combinations thereof.

10. The immunogenic composition of claim 9, wherein the microparticles comprise PLG microparticles.

11. The immunogenic composition of any one of claims 6 to 10, wherein, the HIV envelope polypeptides comprise a polypeptide selected from the group consisting of: a gp120, a gp140 and a gp160.

12. The immunogenic composition of claim 11, wherein at least one HIV envelope polypeptide comprises a gp140 polypeptide, and said gp140 polypeptide is an oligomeric gp140 (o-gp140).

13. The immunogenic composition of claim 12, wherein the o-gp140 comprises a mutation in the protease cleavage site.

14. The immunogenic composition of any one of the preceding claims, wherein one or more of the HIV envelope polypeptides comprises a deletion in V2 loop, a deletion in V1 loop, a deletion in V3 loop or a combination thereof.

15. The immunogenic composition of any one of the preceding claims, wherein the HIV envelope polypeptides is derived from two or more subtypes selected from the group consisting of: subtypes A, B, C, D, E, F, G, H, J, K and circulating recombinant forms (CRFs).

16. The immunogenic composition of claim 15, wherein the two or more subtypes comprise subtypes A and B.

17. The immunogenic composition of claim 15, wherein the two or more subtypes comprise subtypes A and C.

18. The immunogenic composition of claim 15, wherein the two or more subtypes comprise subtypes B and C.

19. The immunogenic composition of claim 15, wherein the two or more subtypes comprise subtypes B and E.

20. The immunogenic composition of any of one of the preceding claims, wherein one or more of the HIV Env polypeptides are complexed to one or more additional molecules selected from the group consisting of: CD4, a CD4 mimetic, a CCR5 co-receptor or mimetic, tat, other viral proteins, polynucleotide, polypeptide, small molecules and combinations thereof.
